SEQUENCE LISTING (14 sequences)

SEQ ID NO: 1 - DNA, 18 bp, Artificial Sequence
  Description of Artificial Sequence Synthetic primer
gtggggcgttaactgcat

SEQ ID NO: 2 - DNA, 20 bp, Artificial Sequence
  Description of Artificial Sequence Synthetic primer
caggtccctgtcatgcttct

SEQ ID NO: 3 - DNA, 19 bp, Artificial Sequence
  Description of Artificial Sequence Synthetic primer
tgctaggggtggctttagg

SEQ ID NO: 4 - DNA, 23 bp, Artificial Sequence
  Description of Artificial Sequence Synthetic primer
caacagctttgctaagtgctcta

SEQ ID NO: 5 - DNA, 20 bp, Artificial Sequence
  Description of Artificial Sequence Synthetic primer
cgtgctctaccttgcaaaca

SEQ ID NO: 6 - DNA, 20 bp, Artificial Sequence
  Description of Artificial Sequence Synthetic primer
tctcctccagctcctcacat

SEQ ID NO: 7 - DNA, 20 bp, Artificial Sequence
  Description of Artificial Sequence Synthetic primer
tgttgtgcgaaaagaagtgc

SEQ ID NO: 8 - DNA, 20 bp, Artificial Sequence
  Description of Artificial Sequence Synthetic primer
tacaaacacagcctcccaca

SEQ ID NO: 9 - DNA, 18 bp, Artificial Sequence
  Description of Artificial Sequence Synthetic primer
ggttcccgaaaccctgag

SEQ ID NO: 10 - DNA, 21 bp, Artificial Sequence
  Description of Artificial Sequence Synthetic primer
gcagcttgagttaaacgaacg

SEQ ID NO: 11 - DNA, 22 bp, Artificial Sequence
  Description of Artificial Sequence Synthetic primer
ttatcagctgcacatcactcag

SEQ ID NO: 12 - DNA, 21 bp, Artificial Sequence
  Description of Artificial Sequence Synthetic primer
cgagaagacctccttcttcca

SEQ ID NO: 13 - DNA, 20 bp, Artificial Sequence
  Description of Artificial Sequence Synthetic primer
ccaccgatccacacagagta

SEQ ID NO: 14 - DNA, 20 bp, Artificial Sequence
  Description of Artificial Sequence Synthetic primer
ggctcctagcaccatgaaga

(19) United States
(12) Patent Application Publication
(10) Pub. No.: US 2009/0022685 A1
(43) Pub. Date: Jan. 22, 2009

(54) Orthotopic, controllable, and genetically tractable non-human animal model for cancer

(76) Inventors: Scott W. Lowe, Cold Spring Harbor, NY (US); Gregory J. Hannon, Huntington, NY (US); Lars Zender, Huntington, NY (US); Wen Xue, Cold Spring Harbor, NY (US); Ross Dickins, Cold Spring Harbor, NY (US)

Correspondence Address: ROPES & GRAY LLP, PATENT DOCKETING 39/361, 1211 AVENUE OF THE AMERICAS, NEW YORK, NY, 10036-8704, US

(21) Appl. No.: 11/893,611

(22) Filed: Aug. 15, 2007

Related U.S. Application Data
(60) Provisional application No. 60/838,025, filed on Aug. 15, 2006.

(51) Int. Cl. A61K 38/20; A61K 35/12; A01K 67/027; G01N 33/574; A61P 31/00; C12Q 1/02; A61K 31/7088; A61K 31/7105

(52) U.S. Cl. 424/85.2; 424/93.21; 800/10; 800/3; 514/44; 435/7.23; 435/29

(57) ABSTRACT

This invention provides a genetically tractable in situ non-human animal model for hepatocellular carcinoma. The model is useful, inter alia, in understanding the molecular mechanisms of liver cancer, in understanding the genetic alterations (e.g., in oncogenes and tumor suppressor genes) that lead to chemoresistance or poor prognosis, and in identifying and evaluating new therapies against hepatocellular carcinomas. The liver cancer model of this invention is made by altering hepatocytes to increase oncogene expression, to reduce tumor suppressor gene expression or both, preferably by inducible, reversible, and/or tissue specific expression of double-stranded RNA molecules that interfere with the expression of a target gene, and by transplanting the resulting hepatocytes into a recipient non-human animal. The invention further provides a method to treat cancer involving cooperative interactions between a tumor cell senescence program and the innate immune system.

### REFERENCE TO RELATED APPLICATIONS

This application claims the benefit of the filing date under 35 U.S.C. § 119(e) of U.S. Provisional Application No. 60/838,025, filed on Aug. 15, 2006, the entire content of which is incorporated herein by reference.

### GOVERNMENT SUPPORT

Work described herein was funded, in whole or in part, by Grant Numbers CA078544, CA13106, CA87497, and CA105388 from the National Institutes of Health (NIH). The United States government has certain rights in the invention.

### TECHNICAL FIELD OF THE INVENTION

This invention provides a genetically tractable, inducible, reversible, or controllable in situ non-human animal model for human cancer, and specifically liver cancer including hepatocellular carcinoma. The model is useful, inter alia, in understanding the molecular mechanisms of cancer in general, in understanding the genetic alterations that lead to chemoresistance or poor prognosis, and in identifying and evaluating new and conventional therapies against cancers.

### BACKGROUND INFORMATION

Cancer is the second leading cause of death in industrial countries. More than 70% of all cancer deaths are due to carcinomas, i.e., cancers of epithelial organs. Most carcinomas show initial or compulsory chemoresistance. This property makes it very difficult to cure these tumors when they are detected in progressed stages.

For example, primary forms of liver cancers include hepatocellular carcinoma, biliary tract cancer and hepatoblastoma. Hepatocellular carcinoma is the fifth most common cancer worldwide but, owing to the lack of effective treatment options, constitutes the leading cause of cancer deaths in Asia and Africa and the third leading cause of cancer death worldwide. Parkin et al., “Estimating the world cancer burden: Globocan 2000.” Int. J. Cancer 94: 153-156 (2001).

The risk factors for liver cancer include excessive alcohol intake or other toxins, such as iron, aflatoxin B1 and also the presence of other infections such as hepatitis B and C. Alison & Lovell. “Liver cancer: the role of stem cells.” Cell Prolif 38: 407-421 (2005). The only curative treatments for hepatocellular carcinoma are surgical resection or liver transplantation, but most patients present with advanced disease and are not candidates for surgery. To date, systemic chemotherapeutic treatment is ineffective against hepatocellular carcinoma, and no single drug or drug combination prolongs survival. Llovet et al. “Hepatocellular carcinoma.” Lancet 362: 1907-1917 (2003). However, despite its clinical significance, liver cancer is understudied relative to other major cancers.

One of the difficulties in identifying appropriate therapeutics for tumor cells in vivo is the limited availability of appropriate test material. Human tumor lines grown as xenographs are unphysiological, and the wide variation between human individuals, not to mention treatment protocols, makes clinical studies difficult. Consequently, oncologists are often forced to perform correlative studies with a limited number of highly dissimilar samples, which can lead to confusing and unhelpful results.

Non-human animal models provide a useful alternative to studies in humans and to human tumor cell lines grown as xenographs, as large numbers of genetically-identical individuals can be treated with identical regimens. Moreover, the ability to introduce germline mutations that affect oncogenesis into these animals increases the power of the models.

To investigate the basic mechanisms of carcinogenesis and to test new potential cancer agents and therapies, however, realistic carcinoma-non-human animal models are urgently needed. So far there have been two major ways to create carcinoma non-human animal models: (i) the generation of transgenic or chimeric non-human animals that express oncogenes under the control of a tissue specific promoter, and, (ii) carcinomas that were induced by chemical carcinogens. Both approaches have several disadvantages.

Current animal models for cancer are based largely on classical transgenic approaches that direct expression of a particular oncogene to an organ of choice using a tissue specific promoter. See, e.g., Wang et al. “Activation of the Met receptor by cell attachment induces and sustains hepatocellular carcinomas in transgenic mice.” J. Cell Biol. 153: 1023-1034 (2001). Although such models have provided important insights into the pathogenesis of cancer, they express the active oncogene throughout the entire organ, a situation that does not mimic spontaneous tumorigenesis. Moreover, incorporation of additional lesions, such as a second oncogene or loss of a tumor suppressor, requires genetic crosses that are time consuming and expensive, and again produce whole tissues that are genetically altered. Finally, traditional transgenic and knockout strategies do not specifically target liver progenitor cells, which may be the relevant initiators of the disease.

Cancer therapies that directly target oncogenes are based on the premise that cancer cells require continuous oncogenic signaling for survival and proliferation. Non-human animal models expressing oncogenes in genetic backgrounds that lack, or have down-regulated, tumor suppressor genes can thus serve as valuable tools to study tumor initiation, maintenance, progression, treatment and regression. However, responses to the targeting drugs are often heterogeneous, and chemoresistance and other resistance is a problem. Because most anticancer agents were discovered through empirical screens, efforts to overcome resistance are hindered by a limited understanding of why these agents are effective and when and how they become less or non-effective.

Furthermore, although cancer usually arises from a combination of mutations in oncogenes and tumor suppressor genes, the extent to which tumor suppressor gene loss is required for the maintenance of established tumors is poorly understood.

Variations in both non-human animal strains and promoters used to drive expression of oncogenes complicate the interpretation of cancer mechanistics and treatment analyses. Firstly, intercrossing strategies to obtain non-human animals of the desired genetic constellation are extremely time consuming and costly. Secondly, the use of certain cell-selective promoters can result in a cell-bias for tumor initiation. For example, the mouse mammary tumor virus (MMTV) promoter and the Whey Acidic Protein (WAP) promoter are commonly used to model breast cancer development in mice, and yet may not target all subtypes of mammary epithelia, i.e., stem cell and non-stem cells. Finally, a homogenous expression of the respective oncogene in all epithelial cells of an organ creates an unphysiological condition, as tumors are known to originate within genetic-mosaics.

An additional difficulty in identifying and evaluating the efficacy of cancer agents on tumor cells and understanding the molecular mechanisms of the cancers and their treatment in the current non-human animal models in vivo is the limited availability of appropriate material.

RNA interference (RNAi) has been used to silence or inhibit the expression of a target gene. RNAi is a sequence-specific post-transcriptional gene silencing mechanism triggered by double-stranded RNA (dsRNA). It causes degradation of mRNAs homologous in sequence to the dsRNA. The mediators of the degradation are 21-23-nucleotide small interfering RNAs (siRNAs) generated by cleavage of longer dsRNAs (including hairpin RNAs) by DICER, a ribonuclease III-like protein. Molecules of siRNA typically have 2-3-nucleotide 3′ overhanging ends resembling the RNAse III processing products of long dsRNAs that normally initiate RNAi. When introduced into a cell, they assemble an endonuclease complex (RNA-induced silencing complex), which then guides target mRNA cleavage. As a consequence of degradation of the targeted mRNA, cells with a specific phenotype of the suppression of the corresponding protein product are obtained (e.g., reduction of tumor size, metastasis, angiogenesis, and growth rates).

The small size of siRNAs, compared with traditional antisense molecules, prevents activation of the dsRNA-inducible interferon system present in mammalian cells. This helps avoid the nonspecific phenotypes normally produced by dsRNA larger than 30 base pairs in somatic cells. See, e.g., Elbashir et al., Methods 26:199-213 (2002); McManus and Sharp, Nature Reviews 3:737-747 (2002); Hannon, Nature 418:244-251 (2002); Brummelkamp et al., Science 296:550-553 (2002); Tuschl, Nature Biotechnology 20:446-448 (2002); U.S. Application US2002/0086356 A1; WO 99/32619; WO 01/36646; and WO 01/68836.

It is therefore important to use a valid animal model to target distinct genetic pathways, preferably in an inducible, reversible, or controllable manner, and preferably using siRNA to knock-down target gene expression, in order to identify new therapeutics for the treatment of liver cancer.

### SUMMARY OF THE INVENTION

The invention provides in vivo and in vitro systems and methods for the study of the effects of tumorigenesis, tumor maintenance, tumor regression, and altered expression of a gene activity, on the descendants of engineered cells, such as embryonic liver progenitor cells or primary hepatocytes. Such engineered (altered) cells, when introduced into a suitable animal, produce cancers such as liver cancers (e.g., hepatocellular carcinomas).

Although the methods and animal models of the invention are generally applicable to several different types of cancers, liver cancer is used as an example for illustration. It should be understood that the scope of the invention is not limited to liver cancer.

One aspect of the invention relates to a (liver) cancer non-human animal model. The liver cancer model of the invention is generated by altering hepatocytes (e.g., embryonic liver progenitor cells or primary hepatocytes, or in short herein, “hepatocytes”) to increase oncogene expression, and to modulate in a controllable manner tumor suppressor gene expression or function. By using inducible, reversible, or controllable promoters, the expression or function of the tumor suppressor gene, may be turned “on” or “off,” going “up” or “down,” or otherwise modulated, depending on specifically controllable conditions. In a preferred embodiment, the increased expression of the oncogene is constitutive, while the expression of the tumor suppressor gene is controlled so that it can be decreased, restored, or increased in comparison to the basal level in the unaltered host cells (e.g., hepatocytes).

The resulting altered hepatocytes are then transplanted into a recipient non-human animal. In certain embodiments, the transplanting is carried out so that the altered hepatocytes engraft the liver of the animal, and a liver tumor develops there from at least one of the altered hepatocytes. In other embodiments, the altered hepatocytes are transplanted subcutaneously into a non-human animal so as to develop a tumor. Tumors are allowed to develop under appropriate conditions.

In certain embodiments, the spontaneous mutations arising in tumors initiated by different oncogenic lesions using the subject methods are compared to alterations observed in human cancers. A good match indicates the close resemblance of the animal model to real life human cancer.

The non-human animal model of hepatocellular carcinoma embodied herein is useful for identifying molecular targets for drug screening, for identifying interacting gene activities, for identifying and evaluating potential therapeutic treatments and for identifying candidates for new therapeutic treatments. The invention also provides methods and non-human animals produced by the methods that are useful for understanding cancer (e.g., liver cancer) and its treatments, and in particular, for evaluating the effect of tumor suppressor gene expression in tumors, and for identifying and studying inhibitors and activators associated with tumor cell growth and growth inhibition, cell death through apoptotic pathways or senescence, and changes in host innate immune response that affect tumor sensitivity and resistance to certain therapies.

The genetically tractable, controllable, and transplantable in situ cancer model (e.g., liver cancer model) of this invention is characterized by genetically defined carcinomas that are preferably traceable by external fluorescent imaging by, for example, tracking the expression of green fluorescent protein (GFP) or its variants, or luciferase, etc. To further characterize the genetic defects in these tumors, gene expression profiling, e.g., representational oligonucleotide microarray analysis (ROMA), can be used to scan the carcinomas for spontaneous gains and losses in gene copy number. Detecting genomic copy number changes through such high resolution techniques can be useful to identify oncogenes (amplifications or gains) or tumor suppressor genes (deletions or losses). Identification of overlapping genomic regions altered in both human and mouse gene array datasets may further aid in pinpointing of regions of interest that can be further characterized for alterations in RNA and protein expression to identify candidates are most likely contributing to the disease phenotype and to be the “driver gene” for amplification.

Using “forward genetics” in combination with gene expression profiling (e.g., ROMA) and the non-human animal models of this invention, important insights into the molecular mechanisms of carcinogenesis, growth, maintenance, regression and remission can be obtained. The models of the invention can directly evaluate the potency of various oncogenes in producing anti-apoptotic phenotypes, and various tumor suppressor genes in producing apoptotic phenotypes and/or senescent phenotypes. Candidate oncogenes or tumor suppressors can be rapidly validated in the non-human animal model of the invention by overexpression, or by using antagonists (e.g., the various stable RNAi technologies), respectively. The invention is also useful in analyzing and evaluating genetic constellations that confer chemoresistance or poor prognosis. Furthermore, the invention is useful for identifying and evaluating new and conventional therapies for the treatment of carcinomas. Finally, one of the unexpected discovery resulting from the use of the subject methods and animal models—that p53-deficient cancers enter a senescent state upon restoration of p53 function leading to an innate immune response—provides a new avenue for treatment of cancers deficient in tumor suppressor genes.

Exemplary embodiments of the invention are listed below in the following numbered paragraphs:
1. A method for making a liver cancer model, said method comprising:

(a) altering hepatocytes:
(1) so as to be capable of modulated tumor suppressor gene expression, said modulation being effected by a controllable inhibition of the expression or function of a tumor suppressor gene in the hepatocytes, and,
(2) to increase oncogene expression, said expression being effected by transducing an oncogene into the hepatocytes;

(b) transplanting said hepatocytes:
(1) into a recipient non-human animal, wherein the hepatocytes engraft the liver of said animal, and a liver cancer develops from at least one of the altered hepatocytes, or,
(2) subcutaneously into a recipient non-human animal, wherein a hepatocellular cancer develops from at least one of the altered hepatocytes.
2. The method of embodiment 1, wherein the controllable inhibition of the expression or function of the tumor suppressor gene is effected by an antagonist capable of inhibiting the expression or function of the tumor suppressor gene, the antagonist being provided in or added to the hepatocytes.
3. The method of embodiment 2, wherein the antagonist is an antibody specific for a gene product encoded by the tumor suppressor gene, a polynucleotide encoding a dominant negative mutant of a gene product encoded by the tumor suppressor gene, or a viral oncoprotein that specifically inactivates a gene product encoded by the tumor suppressor gene.
4. The method of embodiment 2, wherein the antagonist is an siRNA or a precursor molecule thereof.
5. The method of embodiment 2, wherein the antagonist is synthesized in the hepatocytes under the control of a reversible promoter.
6. The method of embodiment 1, wherein the oncogene is a constitutively active ras oncogene or a constitutively active Akt oncogene.
7. The method of embodiment 4, wherein the siRNA is directed against p53.
8. The method of embodiment 5, wherein said promoter is a Pol II promoter.
9. The method of embodiment 8, wherein the Pol II promoter comprises an LTR promoter or a CMV promoter.
10. The method of embodiment 5, wherein the Pol II promoter is affected by a cis-regulatory enhancer.
11. The method of embodiment 5, wherein the reversible promoter is a tetracyclin-responsive promoter.
12. The method of embodiment 11, wherein the tetracyclin-responsive promoter is a TetON promoter, the transcription from which promoter is activated at the presence of tetracyclin (tet), doxycycline (Dox), or a tet analog.
13. The method of embodiment 11, wherein the tetracyclin-responsive promoter is a TetOFF promoter, the transcription from which promoter is turned off at the presence of tetracyclin (tet), doxycycline (Dox), or a tet analog.
14. The method of embodiment 4, wherein the precursor molecule is a precursor microRNA.
15. The method of embodiment 14, wherein the precursor microRNA (miR) is an artificial miR comprising coding sequence for said siRNA.
16. The method of embodiment 15, wherein the miR comprises a backbone design of microRNA-30 (miR-30).
17. The method of embodiment 15, wherein the miR comprises a backbone design of miR-15a, -16, -19b, -20, -23a, -27b, -29a, -30b, -30c, -104, -132s, -181, -191, -223.
18. The method of embodiment 4, wherein the precursor molecule is a short hairpin RNA (shRNA).
19. The method of embodiment 4, wherein the siRNA or precursor molecule thereof is encoded by a single copy of nucleic acid construct integrated into the genome of the hepatocytes.
20. The method of embodiment 19, wherein the nucleic acid construct further comprises an enhancer for the Pol II promoter.
21. The method of embodiment 1, wherein the hepatocytes are embryonic or primary hepatocytes.
22. The method of embodiment 1, further comprising, in step (a), altering the hepatocytes to express a fluorescent marker gene.
23. The method of embodiment 22, wherein the fluorescent marker gene encodes green fluorescent protein (GFP) or luciferase.
24. The method of embodiment 23, wherein the marker gene is GFP.
25. The method of embodiment 1, wherein the altered hepatocytes are transplanted into the spleen of the recipient non-human animal, and migrate via the portal vein into the liver.
26. The method of embodiment 1, wherein the recipient non-human animal is pre-treated with a liver cell cycle inhibitor.
27. The method of embodiment 26, wherein the liver cell cycle inhibitor is Retrorsine.
28. The method of embodiment 1, wherein the recipient non-human animal is post-treated by several administrations of CCl₄.
29. A non-human animal produced by the method of embodiment 1.
30. A method for determining the effect of increasing the expression of a tumor suppressor gene on the efficacy of a potential therapy or potential therapeutic agent for treating liver cancer, comprising:
   (a) administering to a non-human animal, produced by the method of embodiment 1, the potential therapy or the potential therapeutic agent, under a first condition wherein the expression of the endogenous tumor suppressor gene is decreased from its basal level in the unaltered hepatocytes, and under a second condition wherein the expression of the endogenous tumor suppressor gene is increased from its decreased level; and,
   (b) monitoring and comparing the non-human animal for liver tumor formation or growth under the first condition and the second condition,
   wherein increased time to tumor formation or growth when the expression of the tumor suppressor gene is increased indicates a positive impact of the tumor suppressor gene on the efficacy of the potential therapy or the potential therapeutic agent.
31. The method of embodiment 30, wherein the potential therapy is surgery, chemotherapy, radiotherapy, or combination thereof.
32. A method for determining the effect of increasing the expression of a tumor suppressor gene in treating liver cancer, comprising:
   (a) allowing tumor formation or growth in a non-human animal produced by the method of embodiment 1, wherein the expression of an endogenous tumor suppressor gene is decreased from its basal level in the unaltered hepatocytes;
   (b) increasing the expression of the endogenous tumor suppressor gene from its decreased level in the altered hepatocytes in the non-human animal; and,
   (c) monitoring and comparing the non-human animal for liver tumor growth under conditions (a) and (b),
   wherein reduced tumor growth or tumor remission when the expression of the tumor suppressor gene is increased indicates a positive impact of increasing the expression of the tumor suppressor gene in treating liver cancer.
33. A method for determining the role of a gene in liver tumorigenesis, the method comprising:
   (a) introducing into a non-human animal an altered hepatocyte comprising a nucleic acid construct encoding an antagonist of the gene, wherein the synthesis of said antagonist is controlled by a reversible promoter; and,
   (b) expressing the antagonist such that the altered hepatocyte exhibits decreased expression of the gene as compared to its basal level in the unaltered hepatocyte;
      wherein when the altered hepatocyte gives rise to a transfected tumor cell in vivo indicates that the gene negatively regulates liver tumorigenesis.
34. The method of embodiment 33, wherein the antagonist is an siRNA or precursor molecule thereof.
35. A method for treating a patient having a cancer associated with a deficiency in a tumor suppressor gene, comprising expressing the tumor suppressor gene in the cancer to cause senescence of the majority of the cancer cells.
36. The method of embodiment 35, further comprising the step of stimulating the innate immune system of the patient.
37. The method of embodiment 36, wherein the innate immune system of the patient is stimulated by administering to the patient a pharmaceutical composition comprising one or more chemokines.
38. The method of embodiment 37, wherein the chemokines are CSF1, MCP1, IL-15, or CXCL1.
39. The method of embodiment 36, wherein macrophages or neutrophils of the innate immune system are activated or stimulated.
40. The method of embodiment 35 or 36, further comprising administering to the patient an angiogenesis inhibitor.
41. The method of embodiment 35, wherein the tumor suppressor gene is p53.
42. The method of embodiment 41, wherein p53 is expressed transiently.
43. The method of embodiment 41, wherein p53 expression is effected by administering to the patient a pharmaceutical composition comprising a compound that reactivates the tumor suppressor function of p53.
44. The method of embodiment 43, wherein the compound completely or partially restores or increases the transcriptional activation function of a mutant p53 impaired for transcriptional activation, or inhibits wild-type p53 turn-over by MDM2.
45. The method of embodiment 35, wherein the cancer is liver cancer.
46. The method of embodiment 35 or 41, wherein the cancer is associated with a constitutively active ras oncogene or a constitutively activated Akt oncogene.
47. An in vitro assay system comprising a co-culture of:

(a) liver tumor cells having:
(1) modulated tumor suppressor gene expression, said modulation being effected by a controllable inhibition of the expression or function of an endogenous tumor suppressor gene in the liver tumor cells, and,
(2) increased oncogene expression effected by a transduced oncogene; and,

(b) innate immune system cells.
48. The in vitro assay system of embodiment 47, wherein said innate immune system cells comprise macrophages or neutrophils.
49. The in vitro assay system of embodiment 48, wherein said macrophages or neutrophils are stimulated by one or more cytokines.
50. The in vitro assay system of embodiment 47, wherein said liver tumor cells are capable of entering senescence upon restoration of the expression or function of the tumor suppressor gene.
51. A screening method to identify a compound that modulates the interaction between innate immune system cells and senescent liver tumor cells, the method comprising:
   (a) providing a co-culture of the in vitro assay system of embodiment 47;
   (b) contacting the co-culture with a candidate compound; and,
   (c) determining the degree of elimination/killing effect of the senescent liver tumor cells by the innate immune system cells, in the presence and absence of the candidate compound;
   wherein an increase (or decrease) of the degree in the presence of the candidate compound indicates that the candidate compound is a positive (or negative) modulator of the interaction between the innate immune system cells and the senescent liver tumor cells.
52. The screening method of embodiment 51, further comprising inducing, in step (a), the liver tumor cells to undergo senescence by restoring the expression or function of the endogenous tumor suppressor gene.
53. The screening method of embodiment 51, further comprising identifying a binding partner of the compound identified as positive (or negative) modulator in step (c), in either the innate immune system cells or the liver tumor cells.
54. The screening method of embodiment 51, further comprising determining the general toxicity of the compound identified in step (c) to eliminate non-specific modulators.
55. The screening method of embodiment 51, wherein the candidate compound is a polynucleotide vector expressing a candidate product in the liver tumor cells.
56. The screening method of embodiment 51, wherein the candidate product is an siRNA or a precursor molecule thereof.
57. The screening method of embodiment 51, wherein the candidate product is a protein.
58. The screening method of embodiment 51, wherein the candidate compound is from a library of candidate compounds.

### BRIEF DESCRIPTION OF THE DRAWINGS

FIGS. 1A-1E show the generation of p53-deficient liver tumors using conditional RNAi. In FIG. 1 Å, murine embryonic liver progenitor cells (“hepatocytes”) were purified from fetal liver, transduced with retrovirus, and transplanted into the liver of recipient mice via intra-splenic injection. After tumor onset, p53 expression can be restored by doxycycline (Dox) treatment. FIG. 1B shows the maps of the several retroviral vector used in the experiments. FIG. 1C shows restoration of p53 expression by Dox treatment. Protein lysates from cultured liver progenitor cells expressing Ras and Tet-off p53 shRNA were immunoblotted for p53, Ras and Tubulin (as a loading control). FIG. 1D shows liver progenitor cells co-expressing Ras, Tet-off p53 shRNA and a luciferase reporter produced invasive liver tumors in recipient mice. A representative mouse was imaged 5, 9 and 13 days post liver seeding. Color bar represents the intensity of luciferase signal. FIG. 1E is imaging and histopathology of liver tumors. The explanted liver from the animal in FIG. 1D (“Ras”) was imaged for GFP and Luciferase to visualize in situ liver tumors. H&E staining reveals histopathology of invasive hepatocarci-nomas. “V” is a control animal receiving wild-type liver cells infected with empty vectors.

FIGS. 2A-2F show that sustained or brief reactivation of p53 produces complete tumor regression. FIG. 2A shows that sustained reactivation of p53 by Dox-treatment leads to rapid tumor regression. A representative mouse seeded with liver progenitor cells co-expressing Ras, Tet-off shp53 and luciferase was imaged at the indicated time. Dox-treatment was started on day 0. FIG. 2B shows that reactivation of p53 results in the regression of subcutaneous tumors. 1.5×10⁶ ras-transformed liver cells harboring Tet-off shp53 (TRE.shp53) or a non-regulatable p53 shRNA (MLS.shp53) were subcutaneously injected into nude mice. Values are mean±SD (n=4). FIG. 2C shows that p53 reactivation is rapidly reversed by Dox withdrawal. Liver progenitor cells or tumors as in FIG. 2B were treated with Dox for 4 days, and then switched to Dox-free condition. Protein lysates were immunoblotted for p53 and Tubulin (as a loading control). FIG. 2D shows that brief reactivation of p53 is sufficient to suppress colony formation. Liver progenitor cells as in FIG. 2A were plated at low density and either not treated (p53 off), pulse treated with Dox for 2 or 4 days, or left constantly on Dox (p53 on). Staining was performed 8 and 16 days after plating. FIG. 2E shows that pulse reactivation of p53 for 4 days results in complete regression of tumors in the liver. Recipient mice seeded with the progenitor cells as in FIG. 2A were pulse treated with Dox from day 0 through day 4, and imaged at the indicated time. FIG. 2F shows that brief reactivation of p53 is sufficient to regress subcutaneous tumors. Nude mice harboring progenitor cells as in FIG. 2A were either constantly treated with Dox (p53 on) or briefly treated for 4 days (p53 on 4d/off). Tumor size was revealed by luciferase imaging. D0 was the initial day of Dox treatment.

FIGS. 3A-3E show that p53 reactivation is associated with cellular differentiation and senescence. FIG. 3A shows that p53 reactivation is associated with cellular differentiation. Ras-driven liver tumors before (p53 off) and after Dox treatment (p53 on, 6 days) were subjected to immunohistochemical analysis. Normal liver is shown as control. TUNEL and Ki67 are apoptosis and proliferation markers, respectively. Alpha-fetoprotein (AFP) is an embryonic liver- and liver tumor marker. Cytokeratin 8 and Cytokeratin 7 are markers of differentiated hepatocytes and cholangiocytes, respectively. Inset denotes CK7 positive bile duct cells. FIG. 3B shows immunoblots of cellular differentiation markers in the liver tumors with 0, 4 and 6 days of Dox-treatment. Protein lysate from wild type mouse liver was loaded as control. * denotes a non-specific band. FIG. 3C shows that p53 reactivation results in the accumulation of senescence markers. Protein lysates as in FIG. 3B were immunoblotted for the indicated proteins. FIG. 3D shows that tumors with reactivated p53 show senescence-associated-β-galactosidase (SA-β-Gal) activity. The blue staining in the tumor cryosections reveals senescent in the Dox-treated tumors (p53 on). FIG. 3E shows GFP imaging and whole mount SA-β-Gal staining of liver tumors not treated (p53 off) or treated with Dox (p53 on, d6).

FIGS. 4A-4J show that tumor clearance occurs by provoking an innate immune response. FIG. 4A shows that p53 reactivation induces senescence in vitro. Liver progenitor cells harboring ras and Tet-off shp53 were cultured on Dox for 6 days (p53 on) and stained for SA-β-Gal. FIG. 4B shows that senescent liver cells are growth arrested but remain stable in culture. Progenitor cells as in FIG. 4A were cultured with or without Dox and cell numbers were counted every two days. Values are mean±SD (n=2). FIGS. 4C-4H are H&E stainings revealing immune cell infiltration in the regressing tumors. FIG. 4C shows that Dox untreated control tumors only show histopathology of a proliferating carcinoma. FIG. 4D shows peri-tumoral infiltration (arrow) of polymorphonuclear leukocytes (PMNs). FIG. 4E shows intra-tumoral infiltration of PMNs (arrowhead). FIG. 4F shows that at day 6 of Dox-treatment, the PMNs had spread throughout the tumor. FIG. 4G shows a high magnification view of d6 tumor. FIG. 4H shows that at day 13, the tumor architecture was largely damaged. FIG. 4I shows that p53 reactivation is accompanied by increased expression of leukocyte attracting chemokines by the senescent liver cells. RNA expression levels for the indicated chemokines in tumors or cultured progenitor cells harboring Ras and Tet-off shp53 was quantified by RT-Q-PCR of duplicate samples at indicated time points. FIG. 4J shows that selective blockade of innate immune cells results in delayed tumor regression. Subcutaneous liver carcinomas co-expressing ras and the Tet-off p53 shRNA were treated with Dox to induce tumor regression. The macrophage toxin Gadolinium Chloride (GdCl) and an anti-neutrophil antibody were applied to block the innate immune response. Values are mean±SD (n=4).

FIG. 5 shows that doxycycline-treatment turns off the conditional miR30-based p53 sh RNA. Liver tumors co-expressing ras and the tet-off p53 shRNA were treated with Dox for the indicated number of days and harvested for Northern blot analysis. Probes were designed to identify the p53 microRNA derived from the expression vector and U6 as a loading control.

FIGS. 6A and 6B show that pulse p53 reactivation produces rapid tumor regression and senescence. FIG. 6A shows that brief p53 reactivation is sufficient to trigger tumor regression. Recipient mice injected with the progenitor cells expressing ras, the tet-responsive p53 shRNA, tTA, and a luciferase reporter were either not treated (p53 off) or pulse treated with Dox from day 0 through day 2. Animals were imaged using bioluminescence on the indicated days. FIG. 6B shows SA-β-Gal staining of liver tumors 8 days after a 4-day pulse treatment of Dox. The blue staining in the tumor cryosections reveals senescent tumor cells.

FIGS. 7A and 7B show that p53-induced liver tumor regression is associated with infiltration of innate immune cells. FIG. 7A shows immunofluorescence staining of tumor cryosections with the neutrophil marker NIMP-R14. FIG. 7B shows immunofluorescence staining of tumor cryosections using the macrophage marker CD68. Arrowhead denotes CD68⁺ cells in the “p53 on” tumor.

FIGS. 8A-8E show that p53-induced tumor regression is accompanied by progressive blood vessel damage. FIG. 8A represents H&E staining of Dox-untreated liver tumors (p53 off), showing normal blood vessel structure (upper panel). FIGS. 8B-8E represent Dox-treated tumors, showing a progression of blood vessel damage in the time course after p53 reactivation (see text for details).

FIG. 9 shows that antagonists of innate immune cells do not prevent p53-induced senescence in vivo. p53 reactivated tumors (p53 on) treated with saline, macrophage toxin (GdCl) or anti-neutrophil antibody were stained for SA-β-Gal activity. Tumor specimens were harvested 8 days after the start of Dox treatment. A tumor not treated with Dox (p53 off) was stained as control.

FIG. 10A shows co-culture of macrophages with senescent tumor cells following p53 reactivation. Ras;TRE.shp53;tTA liver tumor cells were treated with Doxycycline for 4 days and then cultured with mouse peritoneal macrophages. Tumor cells are positive for GFP and luciferase (Luc). FIG. 10B is bioluminescence imaging of the co-culture. Duplicate wells are shown. FIG. 10C shows representative microscopic view of the co-culture. Arrows indicate senescent tumor cells (GFP positive) covered by GFP negative macrophages.

### DETAILED DESCRIPTION OF THE INVENTION

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, cell and cancer biology, virology, immunology, microbiology, genetics and protein and nucleic acid chemistry described herein are those well known and commonly used in the art.

The methods and techniques of the present invention are generally performed according to conventional methods well known in the art and as described in various general and more specific references that are cited and discussed throughout the present specification, unless otherwise indicated. See, e.g., Sambrook et al., Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2003); Harlow and Lane, Antibodies: A Laboratory Manual, Cold Spring Harbor Laboratory Press, Cold Spring Harbor, N.Y. (1990); Coffin et al., Retroviruses, Cold Spring Harbor Laboratory Press; Cold Spring Harbor, N.Y. (1997); Bast et al., Cancer Medicine, 5th ed., Frei, Emil, editors, BC Decker Inc., Hamilton, Canada (2000); Lodish et al., Molecular Cell Biology, 4th ed., W. H. Freeman & Co., New York (2000); Griffiths et al., Introduction to Genetic Analysis, 7th ed., W. H. Freeman & Co., New York (1999); Gilbert et al., Developmental Biology, 6th ed., Sinauer Associates, Inc., Sunderland, Mass. (2000); and Cooper, The Cell—A Molecular Approach, 2nd ed., Sinauer Associates, Inc., Sunderland, M A (2000). All of the above and any other publications, patents and published patent applications referred to in this application are specifically incorporated by reference herein.

One aspect of the invention provides a method for making a non-human animal bearing a cancer or predisposed to develop a cancer, using transplanted cells altered in such a way that they have increased oncogene expression, and are capable of having controllable tumor suppressor gene expression, preferably in a temporally- and/or spatially-controlled manner. Preferably, the cancer is liver cancer, breast cancer, blood cancer (e.g., lymphomas, leukemia, etc.), or sarcoma. Preferably, the cancer is liver cancer, such as hepatocellular carcinoma.

Although the description herein frequently uses liver cancer as an example, the method of the invention is not so limited, and it should be understood that the methods of the invention apply to other cancers listed above.

Thus in one embodiment, the invention provides a method for making a liver cancer model, the method comprising: (a) altering hepatocytes: (1) so as to be capable of modulated tumor suppressor gene expression, said modulation being effected by a controllable inhibition of the expression or function of a tumor suppressor gene in the hepatocytes, and, (2) to increase oncogene expression, said expression being effected by transducing an oncogene into the hepatocytes; and, (b) transplanting said hepatocytes: (1) into a recipient non-human animal, wherein the hepatocytes engraft the liver of said animal, and a liver cancer develops from at least one of the altered hepatocytes, or, (2) subcutaneously into a recipient non-human animal, wherein a hepatocellular cancer develops from at least one of the altered hepatocytes.

Any oncogene may be used for the subject method, including without limitation: ras (e.g., H-ras, N-ras, K-ras, v-ras with various constitutively activating mutations, such as the V12 mutation), growth factors (e.g., EGF, PDGF), growth factor receptors (e.g., erbB1-4), signal transducers (e.g., abl, Akt), transcription factors (e.g., myc), apoptosis regulators (e.g., bcl-2), etc. In the preferred embodiments, the oncogene is constitutively active.

Any suitable tumor suppressors may be used for the subject method, including without limitation: p53, BRCA1, BRCA2, APC, p16^{INK4a}, PTEN, NF1, NF2, and RB1.

For liver cancer models, the preferred oncogene is a constitutively active ras, Akt, or myc, and the preferred tumor suppressor gene is p53.

More than one oncogene may be used in a model. More than one tumor suppressor gene may be used in a model.

In certain embodiments, the controllable inhibition of the expression or function of the tumor suppressor gene is effected by an antagonist capable of inhibiting the expression or function of a tumor suppressor gene, the antagonist being provided in or added to the hepatocytes. There are many antagonists that may be used in the instant invention.

In a preferred embodiments, the antagonist for the tumor suppressor gene is an siRNA or a precursor molecule thereof, which may be a short hairpin RNA, or a microRNA precursor. Many microRNA precursors can be used, including without limitation a microRNA comprising a backbone design of miR-15a, -16, -19b, -20, -23a, -27b, -29a, -30b, -30c, -104, -132s, -181, -191, -223. See US 2005-0075492 A1 (incorporated herein by reference).

In a preferred embodiment, artificial miRNA constructs based on miR-30 (microRNA 30) may be used to express precursor miRNA/shRNA from single/low copy stable integration in cells in vivo, or through germline transmission in transgenic animals. For example, Silva et al. (Nature Genetics 37: 1281-88, 2005, incorporated herein by reference) have described extensive libraries of pri-miR-30-based retroviral expression vectors that can be used to down-regulate almost all known human (at least 28,000) and mouse (at least 25,000) genes (see RNAi Codex, a single database that curates publicly available RNAi resources, and provides the most complete access to this growing resource, allowing investigators to see not only released clones but also those that are soon to be released, available at http://codex.cshl dot edu). Although such libraries are driven by Pol III promoters, they can be easily converted to the subject Pol II-driven promoters (see Methods in Dickins et al., Nat. Genetics 37: 1289-95, 2005; also see page 1284 in Silva et al., Nat. Genetics 37: 1281-89, 2005).

In certain embodiments, the subject precursor miRNA cassette may be inserted within a gene encoded by the subject vector. For example, the subject precursor miRNA coding sequence may be inserted within an intron, the 5′- or 3′-UTR of a reporter gene, etc.

The many possible siRNA precursor molecules (e.g., short hairpin double strand RNA, and the microRNA-based RNA precursors) are described in more details in a section below.

Alternatively, the antagonist may be polynucleotides encoding one or more antibodies against a tumor suppressor gene product, or a dominant negative mutant of the tumor suppressor gene product, or in certain cases, viral oncoprotein that specifically inactivates the tumor suppressor gene product, etc. Other methods of RNA interference may also be used in the practice of this invention. See, e.g., Scherer and Rossi, Nature Biotechnology 21:1457-65 (2003) for a review on sequence-specific mRNA knockdown of using antisense oligonucleotides, ribozymes, DNAzymes. See also, International Patent Application PCT/US2003/030901 (Publication No. WO 2004-029219 A2), filed Sep. 29, 2003 and entitled “Cell-based RNA Interference and Related Methods and Compositions.”

The controllable inhibition of the expression of the tumor suppressor gene may be effected by controlling the synthesis of the antagonist in the target cell (e.g., the hepatocytes in the liver cancer model). The synthesis of the antagonist may be effected by a promoter from a construct, such as a viral vector. In certain embodiments, the promoter that drives the expression of the antagonist for the tumor suppressor gene is a RNA Polymerase II promoter (Pol II promoter), optionally under the cis-regulation of one or more enhancers. In general, any Pol II compatible promoters may be used for the instant invention. An exemplary Pol II promoter may comprise an LTR promoter or a CMV promoter.

In certain embodiments, various inducible and reversible Pol II promoters may be used to direct antagonist (e.g., precursor miRNA/shRNA) expression. For example, with respect to an siRNA construct (e.g., one based on shRNA or microRNA, etc.), or any other antagonist, an inducible promoter allows the expression of the siRNA at a desired time. The promoter may also be rendered reversible by, for example, using a tightly regulatable tetracyclin-controllable promoter (infra).

As used herein, “reversible” includes the ability to modulate the increase or decrease of the transcription from a promoter for an unlimited number of times. For example, for a tetracyclin-responsive promoter, adding tetracyclin (tet) or its analog (such as Dox) may turn on the transcription from the promoter, while withdrawing tet reverses the process (i.e., turns off the transcription from the promoter). Adding tet later may yet again turn on the transcription.

For example, in certain embodiments, the tetracyclin-responsive promoter is a TetON promoter, the transcription from which promoter is activated at the presence of tetracyclin (tet), doxycycline (Dox), or a tet analog. In other embodiments, the tetracyclin-responsive promoter is a TetOFF promoter, the transcription from which promoter is turned off at the presence of tetracyclin (tet), doxycycline (Dox), or a tet analog. One section below provides more detailed description for such promoters. These tet systems allow incremental and reversible induction of precursor miRNA/shRNA expression in vitro and in vivo, with no or minimal leakiness in precursor miRNA/shRNA expression.

A number of other inducible/reversible expression systems known in the art and/or described herein may also be used. These inducible promoters include without limitation: a promoter operably linked to a lac operator (LacO), a LoxP-stop-LoxP system promoter, or a GeneSwitch™ or T-REx™ system promoter (Invitrogen).

Furthermore, the antagonist may also be expressed in a tissue-specific manner or a developmental stage-specific manner.

Any tissue specific promoters may be used in the instant invention. Merely to illustrate, Chen et al., (Nucleic Acid Research, Vol. 34, database issue, pages D104-D107, 2006) described TiProD, the Tissue-specific Promoter Database (incorporated herein by reference). Specifically, TiProD is a database of human promoter sequences for which some functional features are known. It allows a user to query individual promoters and the expression pattern they mediate, gene expression signatures of individual tissues, and to retrieve sets of promoters according to their tissue-specific activity or according to individual Gene Ontology terms the corresponding genes are assigned to. The database have defined a measure for tissue-specificity that allows the user to discriminate between ubiquitously and specifically expressed genes. The database is accessible at tiprod.cbi.pku dot edu.cn:8080/index.html. It covers most (if not all) the tissues described above.

Thus in certain embodiments, expression of the subject miRNA/shRNA may be under the control of a tissue specific promoter, such as a promoter that is specific for: liver, pancreas (exocrine or endocrine portions), spleen, esophagus, stomach, large or small intestine, colon, GI tract, heart, lung, kidney, thymus, parathyroid, pineal gland, pituitary gland, mammary gland, salivary gland, ovary, uterus, cervix (e.g., neck portion), prostate, testis, germ cell, ear, eye, brain, retina, cerebellum, cerebrum, PNS or CNS, placenta, adrenal cortex or medulla, skin, lymph node, muscle, fat, bone, cartilage, synovium, bone marrow, epithelial, endothelial, vescular, nervous tissues, etc. The tissue specific promoter may also be specific for certain disease tissues, such as cancers. See Fukazawa et al., Cancer Research 64: 363-369, 2004 (incorporated herein by reference).

A combination of the promoters may also be used to express the antagonist construct. For example, an inducible or reversible antagonist may be expressed in a tissue-specific or developmental stage-specific manner. By using one or more of these promoters, the synthesis of the antagonist, and thus the inhibition of the tumor suppressor gene, may be controlled in an inducible, reversible, tissue-specific, and/or a developmental stage-specific manner.

When the inducible, reversible, tissue-specific, or developmental stage-specific promoters are used to regulate expression, the target cell also comprises any of the necessary elements for these promoters to function properly. For example, in the tetracyclin-responsive system TetON or TetOFF, the cell also expresses tTA or rtTA to facilitate the reversible induction of genes operatively linked to such promoters.

The oncogenes (if not endogenous), tumor suppressor genes (if not endogenous), or the antagonists of the tumor suppressor genes described above may be introduced into a target cell by any suitable molecular biology means, such as germline transmission (e.g., transgene), transfection or electroporation coupled with stable integration, infection by viral vectors, etc.

In a preferred embodiment, the oncogene (if not endogenous), the antagonist for the tumor suppressor gene, and/or the marker gene are transduced into a recipient cell via one or more vectors (such as viral vectors), and are stably integrated into the genome of the recipient cell (such as a hepatocyte). A single copy of each of the oncogene, the antagonist for the tumor suppressor gene, and/or the marker gene is usually sufficient for the subject invention, but multiple copies integrated at the same or different genomic locations are also within the scope of the invention. The copy numbers may be controlled by any standard molecular biology means. For example, for viral infection, controlling the ratio of target recipient cells and the viral vectors may result in different integrated copies of the oncogene, the antagonist for the tumor suppressor gene, and/or the marker gene.

In short term primary culture, hepatocytes can be virally transduced with vectors carrying oncogenes or tumor suppressor genes, or expression cassettes for antagonists (such as short hairpin RNAs) directed against tumor suppressor genes. Such transductions may be effected using standard and conventional protocols. Altered hepatocytes virally transduced with such vector(s) expressing an oncogene and/or a reversible siRNA construct (e.g., a short hairpin RNA-based or a microRNA-based) against a tumor gene (e.g., a tumor suppressor gene or other candidate treatment target genes) may be subsequently transplanted into a recipient non-human animal, wherein the animal develops liver tumors from at least one of the hepatocytes with altered gene expression.

Many established viral vectors may be used to transduce foreign constructs into cells. A section below provides more details regarding the use of such vectors. Primary adult or embryonic hepatocyte cultures can be genetically modified by infection with lentiviral- or retroviral vectors carrying various genetic alterations, including oncogenes, or reversibly expressed siRNAs against tumor suppressor genes. These virally transduced hepatocytes can efficiently engraft the livers of non-human animals after transplantation.

Specifically, after viral transduction, the cells are preferably injected into the spleen or portal vein of the recipient non-human animal, preferably a rodent, and most preferably a mouse. The non-human animal are preferably pretreated with a liver cell cycle inhibitor, such as Retrorsine. Using this approach, the genetically modified or altered hepatocytes migrate via the portal vein into the liver and engraft the organ.

An additional proliferation stimulus to the liver can preferably be given after hepatocyte transplantation by serial administration of CCl₄.

Alternatively, after viral transduction, the cells may be injected subcutaneously into a recipient non-human animal, and a hepatocellular cancer may then develop from at least one of the altered hepatocytes. In certain embodiments, the recipient animal is an immuno-compromised animal, such as a nude mouse (e.g., nu/nu mouse).

To facilitate the monitoring of the formation and progression of the cancer, cells harboring the oncogene and tumor suppressor gene may additionally comprise a marker construct, such as a fluorescent marker construct. The marker construct expresses a marker, such as a green fluorescent protein (or its derivatives BFP, RFP, YFP, etc.) or a luciferase gene, which emits fluorescent light constitutively or under inducible conditions. The marker gene may be separately introduced into the cell harboring the oncogene and tumor suppressor gene (e.g. co-transduced, etc.). Alternatively, the marker gene may be linked to the oncogene or tumor suppressor gene construct, and the marker gene expression may be controlled by a separate translation unit under an IRES (internal ribosomal entry site).

In mice having developed hepatocellular carcinomas and also expressing a fluorescent marker protein (such as GFP) in the carcinoma, tumor progression can be easily visualized by whole body fluorescence imaging. See, e.g., Schmitt et al., “Dissecting p53 tumor suppressor functions in vivo,” Cancer Cell 1:289-98 (2002).

The size and growth of tumors before and/or after therapy can be monitored by any of many ways known in the art. Tumors can also be examined histologically. Paraffin embedded tumor sections can be used to perform immunohistochemistry for cytokeratins and ki-67 as well as TUNEL-staining. The apoptotic rate of hepatocytes can be analyzed by TUNEL assay according to published protocols. Di Cristofano et al., “Pten and p27KIPI cooperate in prostate cancer tumor suppression in the mouse,” Nature Genetics, 27:222-224 (2001).

The genetically tractable, transplantable, controllable in situ liver or hepatocellular cancer model of the invention offers unique advantages. This invention employs the proliferative capacity of the liver to enable the altered hepatocytes to reconstitute liver tissue. Large amounts of primary epithelial cells can be isolated according to standardized protocols either from adult mouse livers or from embryonic mouse livers. The mouse can be either wild-type or harboring one or more transgenes.

Another aspect of the invention provides a non-human animal produced by the method of the subject invention as described herein (see supra). Preferably, the animal is a rodent, such as a mouse or a rat.

In certain embodiments, the pathology of the tumor developed in the animal is determined and/or compared with the corresponding human tumors, in order to verify that the animal model reflects the human disease as near as possible.

Another aspect of the invention provides a method for determining the effect of increasing the expression of a tumor suppressor gene on the efficacy of a potential therapy or potential therapeutic agent in treating liver cancer, comprising: (a) administering to a non-human animal, produced by the subject method, the potential therapy or the potential therapeutic agent, under a first condition wherein the expression of the endogenous tumor suppressor gene is decreased from its basal level in the unaltered hepatocytes, and under a second condition wherein the expression of the endogenous tumor suppressor gene is increased from its decreased level; and, (b) monitoring and comparing the non-human animal for liver tumor formation or growth under the first condition and the second condition, wherein increased time to tumor formation or growth when the expression of the tumor suppressor gene is increased indicates a positive impact of the tumor suppressor gene on the efficacy of the potential therapy or the potential therapeutic agent.

In certain embodiments, the potential therapy is surgery, chemotherapy, radiotherapy, or combination thereof.

One of the recurring problems of cancer therapy is that a patient in remission (after the initial treatment by surgery, chemotherapy, radiotherapy, or combination thereof) may experience relapse. The recurring cancer in those patients is frequently resistant to the apparently successful initial treatment. In fact, certain cancers in patients initially diagnosed with the disease may be already resistant to conventional cancer therapy even without first being exposed to such treatment. Thus there is a need to identify new therapies in these patients in order to treat these resistant cancer.

Many cancers resisting to treatment may contain one or more mutations in tumor suppressor genes, the existence of which may be detected by various standard molecular biology means, such as immunoblotting using antibodies specific for the tumor suppressor gene product, in situ hybridization using a nucleic acid probe specific for the tumor suppressor gene, or direct observation of the diseased chromosomes harboring a deletion or other abnormalities in the chromosomal region where the tumor suppressor gene resides, etc.

Once the presence of the loss of tumor suppressor gene(s) is confirmed, it remains unclear whether in that cancer, continued absence of a specific tumor suppressor gene is required for the resistance to therapy. In certain cancers, restoring the function (e.g., by increasing the expression) of the tumor suppressor gene may have a positive impact on therapy, e.g., it will render the cancer responsive to conventional therapy. In certain other cancers, restoring the function of the tumor suppressor gene would have no appreciable effect on cancer responsiveness to conventional therapy. Thus it is important to determine which category a cancer of interest belongs before devoting time and resources to restore or increase the function of the tumor suppressor gene.

The methods of the instant invention provide a powerful tool to address the question. Applicants have demonstrated that in a liver cancer model, restoring previously suppressed endogenous p53 expression will cause the liver cancer cells to enter a differentiated or senescence state, which triggers the innate immune system of the patient to attack and destroy the cancer cells and their vesculature. Thus, cancer senescence coupled with immune system activation lead to tumor involution in the subject liver cancer model. This unexpected discovery not only verifies that p53 is an effective tumor suppressor gene target for therapeutic intervention, but also demonstrates that, at least in liver cancer, increasing p53 function may render a previously ineffective or less effective immune therapy (e.g., one that stimulates the patient's innate immune response) effective or more effective.

Another aspect of the invention provides a method for determining the effect of increasing the expression of a tumor suppressor gene in treating liver cancer, comprising: (a) allowing tumor formation or growth in a non-human animal produced by the subject method, wherein the expression of an endogenous tumor suppressor gene is decreased from its basal level in the unaltered hepatocytes; (b) increasing the expression of the endogenous tumor suppressor gene from its decreased level in the altered hepatocytes in the non-human animal; and, (c) monitoring and comparing the non-human animal for liver tumor growth under conditions (a) and (b), wherein reduced tumor growth or tumor remission when the expression of the tumor suppressor gene is increased indicates a positive impact of increasing the expression of the tumor suppressor gene in treating liver cancer.

As described above, certain cancer patients may have lost tumor suppressor genes in their cancers, and it is important to determine whether restoring or increasing the function of such tumor suppressor genes would be an effective therapy for such patients. The methods and animal models of the invention provides a powerful tool to address this problem, by allowing one to create a cancer lacking functional expression of one or more tumor suppressor genes, then monitoring the progression of that cancer after restoring or increasing the expression of the previously missing tumor suppressor gene. If restoring or increasing the expression of the tumor suppressor gene delays or even reverses cancer progression, the tumor suppressor gene is a valid target for therapeutic intervention, and it is justified to devote time and resource to develop therapies to restore or increase the expression of the tumor suppressor gene in such patients.

Once a tumor suppressor gene has been validated as a potential target, the increased expression of which in a cancer has been shown to be able to delay or even reverse the progression of the cancer, the invention also provides a method to treat that cancer, comprising increasing the expression of the tumor suppressor gene in the cancer (which has decreased or depressed expression of the tumor suppressor gene).

In certain embodiments, the tumor suppressor gene is p53.

Another aspect of the invention provides a method for determining the effect of decreasing the expression or function of a candidate endogenous gene in treating liver cancer, comprising: (a) allowing tumor formation or growth in a non-human animal produced by the subject method, wherein the expression or function of the candidate endogenous gene is capable of being decreased from its basal level in the tumor; (b) decreasing the expression or function of the candidate endogenous gene from its basal level in the tumor; and, (c) monitoring and comparing the non-human animal for liver tumor growth under conditions (a) and (b), wherein reduced tumor growth or tumor remission when the expression or function of the candidate endogenous gene is decreased indicates that the candidate endogenous gene is a valid target for treating liver cancer.

This aspect of the invention provides an effective means to determine whether inhibition of a candidate endogenous gene would be a valid approach for cancer therapy, such that small molecule inhibitors or other inactivating approaches should be pursued. The method of the invention can be used to validate cancer therapy targets, no only for the oncogenes or tumor suppressor genes that cause or lead to the initial tumorigenesis, but also for any endogenous candidate gene whose expression or function is possibly required to maintain tumor growth or progression. These candidate genes may be any relevant genes, such as downstream targets for the oncogenes, or inhibitors of the tumor suppressor genes, or regulators of the oncogenes or tumor suppressor genes that cause the initial tumorigenesis, etc. As described above, the expression or function of such candidate genes may be modulated by an antagonist at any desired stages after the initial tumorigenesis, to study whether continued expression or function of that gene is required for maintaining tumor growth or progression, including invasion and metastasis, and if so, during and by what stage.

The antagonist can be any of the antagonists described herein, such as the various siRNA constructs (e.g., shRNA-based or microRNA-based), antisense polynucleotides, antibodies against the gene products, dominant negative mutants, etc. For example, during initial tumorigenesis, an antagonist of a candidate gene (such as a microRNA-based siRNA construct) may be controlled by the Tet-responsive system described herein, such that no siRNA is produced. As tumorigenesis progress, the expression of the siRNA may be turned on or up-regulated, so as to partially or completely down-regulate the expression or function of the candidate gene.

In certain embodiments, the basal expression level of the candidate gene may be up-regulated in the tumor (for example, when the candidate gene is a downstream target of the oncogene). Alternatively, the gene product of the candidate gene may switch from an inactivated form (e.g., unphosphorylated form) to an activated form (e.g., phosphorylated form). In either circumstances, the antagonists may be induced to be expressed at a desired time to down-regulate the functional form of the candidate gene, in order to assess the effect of decreasing the expression or function of the candidate endogenous gene in treating liver cancer.

Another aspect of the invention provides a method for determining the role of a gene in liver tumorigenesis, the method comprising: (a) introducing into a non-human animal an altered hepatocyte comprising a nucleic acid construct encoding an antagonist of the gene, wherein the synthesis of said antagonist is controlled by a controllable promoter; and, (b) effect the expression of the antagonist, if necessary, such that the altered hepatocyte exhibits decreased expression of the gene; wherein the altered hepatocyte gives rise to a transfected tumor cell in vivo indicates that the gene negatively regulates liver tumorigenesis.

According to this aspect of the invention, the expression of a candidate tumor suppressor gene is turned off in a hepatocyte via, for example, an siRNA construct (supra). If an animal having the altered hepatocyte develops an in vivo cancer, the function of the tumor suppressor gene is required to suppress (or negatively regulate) liver cancer formation.

In fact, the method of the invention may generally be applied to any of the other cancers. For example, if it is found, using the subject reversible inhibition system, that turning off a candidate tumor suppressor gene promotes tumorigenesis of a particular cancer in an animal model, the tumor suppressor gene is a valid intervention target for treating that particular cancer.

Although the same result may be achieved using conventional gene knock-out technology, the method of the instant invention provides a distinct advantage in allowing one to subsequently turn back on the expression of the tumor suppressor gene, and monitor the progression of the cancer, now at the presence of the functional tumor suppressor gene. Thus the systems, methods, and animal models of the invention not only effectively addresses the question of whether a particular tumor suppressor gene is important for suppressing tumorigenesis of a particular cancer, but also addresses the independent question of whether, after the initiations of tumorigenesis, restoring or increasing the expression of the tumor suppressor gene has a positive impact for cancer therapy.

Another aspect of the invention provides a method for treating a patient having a cancer associated with a deficiency in a tumor suppressor gene, comprising expressing the tumor suppressor gene in the cancer to cause senescence of the majority of the cancer cells.

As used herein, “majority” refers to a level no less than 50%, or 60%, 70%, 80%, 90%, 95%, 99%, or close to 100%.

In certain embodiments, the method further comprises the step of stimulating the innate immune system of the patient.

This aspect of the invention is partly based on the surprising discovery that restoring p53 expression in certain p53-deficient cancers, such as a p53-deficient liver cancer, causes the cancer cells to differentiate or to senesce (rather than to become apoptotic). These cancer cells was found to produce certain leukocyte-attracting chemokines that attract cells of the innate immune system, such as polymorphonuclear leukocytes (PMNs) including neutrophils, and macrophages. These cells in turn attack the senesced tumor cells as well as the tumor vesculature to destroy the tumor.

Thus the invention provides a method to boosting the immune response of a cancer patient, especially the innate immune response of the patient, in conjunction with a therapy to increase tumor suppressor gene expression in a cancer deficient for tumor suppressor gene expression. In certain embodiments, the tumor suppressor gene is p53.

In certain embodiments, the innate immune system of the patient is stimulated by administering to the patient a pharmaceutical composition comprising one or more chemokines that stimulate macrophages and/or polymorphonuclear leukocytes (PMNs) including neutrophils, or promotes the proliferation and/or differentiation of macrophages and/or neutrophils. Exemplary chemokines (without limitation) include CSF1 (Colony-Stimulating Factor 1), MCP1 (Monocyte Chemotactic Protein-1), IL-15 (Interleukin-15), or CXCL1 (CXC Motif Chemokine Ligand 1).

In certain embodiments, therapy may further comprise administering to the patient an angiogenesis inhibitor, such as thrombospondin 2 (THBS2) and thrombospondin 4 (THBS4).

According to this aspect of the invention, even temporary restoration of p53 function in p53-deficient cancers is sufficient to trigger the cancer cells to enter the senescence state. Thus in certain embodiments, the method comprising restoring or increasing the function of p53 only transiently. This may be desirable, because of the obvious advantages of lesser cost in medical care and reduced patient suffering. It may also be desirable, since stable expression of a gene (such as p53) frequently requires the use of viral vectors to infect cells of a cancer patient. The integration of such viral vectors into the host genome is usually not precisely controlled. Thus there is a risk that insertion of the viral vectors into the host genome may inadvertently causing damages to the host cell, including healthy cells that happen to receive a viral infection. Another potential problem with the stable integration of such viral vectors includes the unpredictability of long-term unnatural expression of a tumor suppressor gene.

The methods of the instant invention enables the use of technology that effects a pulse expression of certain tumor suppressor genes, such as transient expression without host genome integration. In certain embodiments, tumor suppressor gene products (i.e., proteins) may also be delivered directly to the tumor via, for example, peptide-mediated transcytosis (see, e.g., U.S. Pat. Nos. 4,992,255, 5,254,342, and 6,204,054) or liposome-mediated protein delivery (see, for example, U.S. Pat. No. 6,420,411).

In certain embodiments, expression of the functional p53 is effected by administering to the patient a pharmaceutical composition comprising a compound that reactivates the tumor suppressor function of p53. For example, the compound may function to completely or partially restore or increase the transcriptional activation function of a mutant p53 impaired for transcriptional activation, or to inhibit wild-type p53 turn-over by MDM2.

As described above, one surprising discovery made using the subject experimental system is that restoring endogenous p53 function triggers cellular senescence and activation of host innate immune response in p53-deficient tumors. This finding provides a new therapeutic avenue for treating p53-deficient tumors.

As used herein, “p53-deficient” refers to that fact that functional p53 expression is less than wild-type level, including complete or partial loss of wild-type p53 expression, due to, for example, mutation or increased degradation of wild-type p53. When there is a p53 mutation in the cell, however, the cell may still express a mutant version of p53 that does not possess the wild-type p53 function, such as its transcriptional activity or apoptosis-inducing activity. The mutant p53 may be a dominant negative p53, or a defective protein with no apparent function.

The most common target for mutations in tumors is the p53 gene. The fact that around half of all human tumors carry mutations in this gene is solid testimony as to its critical role as a tumor suppressor. p53 halts the cell cycle and/or triggers apoptosis in response to various stress stimuli, including DNA damage, hypoxia, and oncogene activation (Ko and Prives, 1996; Sherr, 1998). Upon activation, p53 initiates the p53 dependent biological responses through transcriptional transactivation of specific target genes carrying p53 DNA binding motifs. In addition, the multifaceted p53 protein may promote apoptosis through repression of certain genes lacking p53 binding sites, and transcription-independent mechanisms as well (Bennett et al., 1998; Gottlieb and Oren, 1998; Ko and Prives, 1996). Analyses of a large number of mutant p53 genes in human tumors have revealed a strong selection for mutations that inactivate the specific DNA binding function of p53; most mutations in tumors are point mutations clustered in the core domain of p53 (residues 94-292) that harbors the specific DNA binding activity (Beroud and Soussi, 1998).

Both p53-induced cell cycle arrest and apoptosis could be involved in p53-mediated tumor suppression. Taking into account the fact that more than 50% of human tumors carry p53 mutations, it appears highly desirable to restore the function of wild type p53-mediated growth suppression to tumors. The advantage of this approach is that it will allow selective elimination of tumor cells carrying mutant p53. Tumor cells are particularly sensitive to p53 reactivation, since, inter alia, mutant p53 proteins tend to accumulate at high levels in tumor cells. Therefore, restoration of the wild type function to the abundant and presumably “inactivated” mutant p53 should trigger a massive response in already sensitized tumor cells, whereas normal cells that express low or undetectable levels of p53 should not be affected or less affected. The feasibility of p53 reactivation as an anticancer strategy is supported by the fact that a wide range of mutant p53 proteins are susceptible to reactivation. A therapeutic strategy based on rescuing p53-induced apoptosis should therefore be both powerful and widely applicable.

For the above defined purpose, it has been shown that p53 is a specific DNA binding protein, which acts as a transcriptional activator of genes that control cell growth and death. Thus, the function of the wild-type p53 protein is largely dependent on its specific DNA binding function. Mutant p53 proteins carrying amino acid substitutions in the core domain of p53, which abolish the specific DNA binding, are non-functional (e.g., unable to induce apoptosis) in cells. Therefore, in order to obtain small molecules capable of restoring p53 function, reactivation of p53 specific DNA binding may be important to trigger p53-dependent function in tumors during pathological conditions.

Many small molecule compounds have been screened and identified to have the capability to restore wild-type p53 function completely or partially. For example, WO 03/070250 A1 describes the screening for and identification of 2 families of compounds, namely 2,2-bis(hydroxymethyl)-1-azabicyclo(2.2.2)octan-3-one and 1-(propoxymethyl)-maleimide, that are capable of reactivating p53 function, through restoration of sequence-specific DNA-binding activity and transcriptional transactivation function to mutant p53 proteins, and modulation of the conformation-dependent epitopes of the p53 protein.

Thus the instant invention provides a method to screening for small molecules capable of restoring mutant p53 function, comprising contacting a proliferating cell expressing a mutant p53 (and optionally an oncogene) with a candidate compound, and determining the presence of one or more senescence markers including (without limitation) p15^{INK4a}, DcR2, p15^{INK4b}, and senescence-associated β-galactosidase (SA-β-Gal), or determining the presence of senescence phenotype/morphology.

An alternative small molecule screening relates to the small molecule to inactivate MDM2. MDM2 binds the p53 tumor suppressor protein with high affinity and negatively modulates its transcriptional activity and stability. Overexpression of MDM2, found in many human tumors, effectively impairs wild-type p53 function. Inhibition of MDM2-p53 interaction can stabilize p53, and effectively restores wild-type p53 function in MDM2-overexpressing cells. Potent and selective small-molecule antagonists of MDM2 have been identified, which bind MDM2 in the p53-binding pocket and activate the p53 pathway in cancer cells, leading to cell cycle arrest, apoptosis, and growth inhibition of human tumor xenografts in nude mice (Vessilev et al., Science 303: 844-8, 2004).

Thus the instant invention provides a method to screening for small molecules capable of restoring wild-type p53 function in MDM2-overexpressing cells, comprising contacting a proliferating cell overexpressing MDM2 and a wild-type p53 (and optionally an oncogene) with a candidate compound, and determining the presence of one or more senescence markers including (without limitation) p15^{INK4a}, DcR2, p15^{INK4b}, and senescence-associated β-galactosidase (SA-β-Gal), or determining the presence of senescence phenotype/morphology.

In certain embodiments, the cancer treatable by the subject method is not only deficient for p53, but also associated with a constitutively active ras oncogene or a constitutively activated Akt oncogene (infra).

In accordance with this invention, it was demonstrated for the first time that senescent cells, such as p53-deficient cancer cells restored for p53 expression, can be eliminated or cleared in vivo and in vitro, partly through a mechanism involving the stimulation of the innate immune system, including macrophages and polymorphonuclear leukocytes (PMNs) including neutrophils. Without wishing to be bound by any particular theory, the mechanism may also involve up-regulation of certain molecules, such as cell surface adhesion molecules in tumor cells undergoing senescence. Exemplary adhesion molecules include ICAM1, VCAM1, NCAM, etc.

Another aspect of the invention provides an in vitro assay system comprising a co-culture of: (a) (liver) tumor cells having: (1) modulated tumor suppressor gene expression, said modulation being effected by a controllable inhibition of the expression or function of an endogenous tumor suppressor gene in the (liver) tumor cells, and, (2) increased oncogene expression effected by a transduced oncogene; and, (b) innate immune system cells.

In certain embodiments, the innate immune system cells comprise macrophages or neutrophils. The macrophages or neutrophils may be stimulated by one or more cytokines, such as CSF1, MCP1, CXCL1, and/or IL15.

In certain embodiments, the (liver) tumor cells are capable of entering senescence upon restoration of the expression or function of the tumor suppressor gene, such as p53.

Another aspect of the invention provides a screening method to identify a compound that modulates the interaction between innate immune system cells and senescent (liver) tumor cells, the method comprising: (a) providing a co-culture of the subject in vitro assay system; (b) contacting the co-culture with a candidate compound; and, (c) determining the degree of elimination/killing effect of the senescent (liver) tumor cells by the innate immune system cells, in the presence and absence of the candidate compound; wherein an increase (or decrease) of the degree in the presence of the candidate compound indicates that the candidate compound is a positive (or negative) modulator of the interaction between the innate immune system cells and the senescent (liver) tumor cells.

In certain embodiments, the screening method further comprises inducing, in step (a), the (liver) tumor cells to undergo senescence by restoring the expression or function of the endogenous tumor suppressor gene.

In certain embodiments, the screening method further comprises identifying a binding partner of the compound identified as positive (or negative) modulator in step (c), in either the innate immune system cells or the (liver) tumor cells. Numerous art-recognized methods may be used to identify binding partners of a compound, such as a protein or small molecule. Such methods include, for example, two- or three-hybrid screening methods, phage display, in vitro binding assay, etc.

In certain embodiments, the screening method further comprises determining the general toxicity of the compound identified in step (c) to eliminate non-specific modulators. This may be advantageous since certain compounds identified in the screen may be generally toxic to all cells, including tumor cells in the assay. It may be desirable to eliminate such generically toxic compounds from the screen.

Any compounds may be used as candidate compounds for the subject method. In certain embodiments, the candidate compound may be a polynucleotide vector expressing a candidate product in the (liver) tumor cells. For example, a library of vectors, each encoding a different product, may be transfected/infected into the tumor cells to express the product. In the case where the product is an siRNA or a precursor molecule thereof, it may down-regulate one or more target genes in the tumor cells, such as an activated oncogene. In the cases where the product is a protein, it may be a cell surface adhesion molecule that becomes expressed in senescent tumor cells, or may be a signaling molecule that triggers the senescence program inside the tumor cell, etc.

In certain embodiments, the candidate compound is from a library of candidate compounds, which may be used in the subject screening methods, preferably in a high-through-put fashion. For example, multiple-well plates may be set up, each well having a subject co-culture, and each well receiving a different candidate compound from the library. Since the tumor cells may be labeled by a fluorescent or bioluminescent marker (GFP, luciferase, etc.), the amount of fluorescence or bioluminescence may be determined in high throughput fashion using, for example, a fluorescent or bioluminescent plate reader.

As used herein, “a non-human animal” includes any animal, other than a human. Examples of such non-human animals include without limitation: aquatic animals, e.g., fish, sharks, dolphins and the like; farm animals, e.g., pigs, goats, sheep, cattle, horses, rabbits and the like; rodents, e.g., rats, hamsters, guinea pigs, and mice; non-human primates, e.g., baboons, chimpanzees and monkeys; and domestic animals, e.g., cats and dogs. Rodents are preferred. Mice are more preferred.

The non-human animals can be wild-type or can carry genetic alterations. For example, they may be immuno-compromised or immuno-deficient, e.g., a severe combined immunodeficiency (SCID) animal. They may also harbor one or more germ-line transgenes, which may be expressed in a tissue-specific and/or developmental stage-specific manner, or ubiquitously expressed.

As used herein, “hepatocytes” include all descendants of embryonic liver progenitor cells and primary hepatocytes. In certain embodiments, primary hepatocytes are used in the methods and models of this invention. Primary hepatocytes from adult non-human animals or embryonic liver progenitor cells can be isolated using standard and conventional protocols.

The primary culture conditions for embryonic as well as adult primary hepatocytes are based on well-established protocols, and are less complex compared to other epithelial primary cultures. A sample of the primary cells can be used for RT-PCR characterization for liver specific markers to rule out overgrowing by non-parenchymal cells.

The term “vector” refers to a nucleic acid molecule capable of transporting another nucleic acid to which it has been linked. One type of vector is a plasmid, which refers to a circular double stranded DNA loop into which additional DNA segments may be ligated. A preferred type of vector for use in this application is a viral vector, wherein additional DNA segments may be ligated into a viral genome that is usually modified to delete one or more viral genes. Certain vectors are capable of autonomous replication in a host cell into which they are introduced (e.g., vectors having an origin of replication which functions in the host cell). Other vectors can be integrated stably into the genome of a host cell upon introduction into the host cell, and are thereby replicated along with the host genome.

Preferred viral vectors include retroviral and lentiviral vectors. For example, stable precursor miRNA/shRNA expression may be effected through retroviral or lentiviral delivery of the miRNA/shRNAs, which is shown to be effective at single copy per cell. This allows very effective stable gene expression regulation at extremely low copy number per cell (e.g. one per cell), thus vastly advantageous over systems requiring the introduction of a large copy number of constructs into the target cell by, for example, transient transfection.

Moreover, certain preferred vectors are capable of directing the expression of nucleic acid sequences to which they are operatively linked. Such vectors are referred to herein as recombinant expression vectors or simply, expression vectors.

Preferably, the vector carries marker cassettes, more preferably, GFP or luciferase expression cassettes, so that the course of transduction, engrafting and tumor growth and remission may be easily observed. Preferably, the vector also carries a drug selective marker gene, such as the neomycin, hygromycin, puromycin resistance genes, etc. Preferably, the vector also carries an enhancer. Preferably, the vector also carries a transcriptional termination signal. Preferably, the vector also carries a second transcription unit with an internal ribosomal entry site (IRES).

Preferably, the vector also carries a promoter, such a ubiquitous promoter that permit expression or up-regulation of oncogenes in all cell types of epithelium (i.e., stem cell and non-stem cell compartments); or an inducible, reversible, tissue-specific, or developmental-stage-specific promoter.

As used herein, “viral transduction” refers to a general method of gene transfer. As embodied herein, viral transduction is used for establishing stable expression of genes in culture. Viral transduction and long-term expression of genes in cells, preferably cultured hepatocytes, is preferably accomplished using viral vectors.

As used herein, an “altered hepatocyte” refers to a change in the level of a gene and/or gene product with respect to any one of its measurable activities in a hepatocyte (e.g., the function which it performs and the way in which it does so, including chemical or structural differences and/or differences in binding or association with other factors). An altered hepatocyte may be effected by one or more structural changes to the nucleic acid or polypeptide sequence, a chemical modification, an altered association with itself or another cellular component or an altered subcellular localization. Preferably, an altered hepatocyte may have “activated” or “increased” expression of an oncogene, “repressed” or “decreased” expression of a tumor suppressor gene, or both.

The “increased expression of an oncogene” refers to a produced level of transcription and/or translation of a nucleic acid or protein product encoded by an oncogenic sequence in a cell. Increased expression or up-regulation of an oncogene can be non-regulated (i.e., a constitutive “on” signal) or regulated (i.e., the “on” signal is induced or repressed by another signal or molecule within the cell). An activated oncogene can result from, e.g., over expression of an encoding nucleic acid, an altered structure (e.g., primary amino acid changes or post-transcriptional modifications such as phosphorylation) which causes higher levels of activity, a modification which causes higher levels of activity through association with other molecules in the cell (e.g., attachment of a targeting domain) and the like.

The decreased expression of a tumor suppressor gene refers to an inhibited, inactivated or down regulated level of transcription and/or translation of a nucleic acid or protein product encoded by a tumor suppressor gene sequence in a cell. Reduced expression of a tumor suppressor gene can be non-regulated (i.e., a constitutive “off” signal) or regulated (i.e., the “off” signal is activated or repressed by another signal or molecule within the cell). As preferred herein, a repressed tumor suppressor gene can result from inhibited expression of an encoding nucleic acid (e.g., most preferably a short hairpin RNA or microRNA using RNA interference approaches). Reduced expression of a tumor suppressor gene can also result from an altered structure (e.g., primary amino acid changes or post-transcriptional modifications such as phosphorylation) which causes reduced levels of activity, a modification which causes reduced levels of activity through association with other molecules in the cell (e.g., binding proteins which inhibit activity or sequestration) and the like.

A “short hairpin RNA (shRNA)” refers to a segment of RNA that is complementary with a portion of one or more target genes (i.e. complementary with one or more transcripts of one or more target genes). When a nucleic acid construct encoding a short hairpin RNA is introduced into a cell, the cell incurs partial or complete loss of expression of the target gene. In this way, a short hairpin RNA functions as a sequence-specific expression inhibitor or modulator in transfected cells. The use of short hairpin RNAs facilitates the down-regulation of tumor suppressor genes and allows for analysis of hypomorphic alleles. The short hairpin RNAs that are useful in the invention can be produced using a wide variety of RNA interference (“RNAi”) techniques that are well known in the art. The invention may be practiced using short hairpin RNAs that are synthetically produced as well as microRNA (miRNA) molecules that are found in nature and can be remodeled to function as synthetic silencing short hairpin RNAs. In a preferred embodiment of the invention, a microRNA-based siRNA precursor mediates inducible and reversible inhibition of a tumor suppressor gene. Preferably, the siRNA or precursor thereof is against p53.

As used herein, the term “liver or hepatocellular cancer/tumor” refers to a group of cells or tissue which are committed to a hepatocellular lineage, and which exhibit an altered growth phenotype. The term encompasses tumors that are associated with hepatocellular malignancy (i.e., HCC), as well as with pre-malignant conditions such as hepatoproliferative and hepatocellular hyperplasia, and hepatocellular adenoma, which include proliferative lesions that are perceived to be secondary responses to degenerative changes in the liver.

As used herein, the terms “cancer” or “tumor” are used interchangeably.

Throughout this specification and embodiments, the word “comprise” or variations such as “comprises” or “comprising”, will be understood to imply the inclusion of a stated integer or group of integers but not the exclusion of any other integer or group of integers.

As described above, many siRNA precursor molecules may be used in the instant invention. The following section provide more details regarding certain preferred siRNA precursors, such as the microRNA-based siRNA precursors.

DNA vectors that express perfect complementary short hairpins RNAs (shRNAs) are commonly used to generate functional siRNAs. However, the efficacy of gene silencing mediated by different short-hairpin derived siRNAs may be inconsistent, and a substantial number of short-hairpin siRNA expression vectors can trigger an anti-viral interferon response (Nature Genetics 34: 263, 2003). Moreover, siRNA short-hairpins are typically processed symmetrically, in that both the functional siRNA strand and its complement strand are incorporated into the RISC complex. Entry of both strands into the RISC can decrease the efficiency of the desired regulation and increase the number of off-target mRNAs that are influenced. In comparison, endogenous microRNA (miRNA) processing and maturation is a fairly efficient process that is not expected to trigger an anti-viral interferon response. This process involves sequential steps that are specified by the information contained in miRNA hairpin and its flanking sequences.

MicroRNAs (miRNAs) are endogenously encoded ˜22-nt-long RNAs that are generally expressed in a highly tissue- or developmental-stage-specific fashion and that post-transcriptionally regulate target genes. More than 200 distinct miRNAs having been identified in plants and animals, these small regulatory RNAs are believed to serve important biological functions by two prevailing modes of action: (1) by repressing the translation of target mRNAs, and (2) through RNA interference (RNAi), that is, cleavage and degradation of mRNAs. In the latter case, miRNAs function analogously to small interfering RNAs (siRNAs). Importantly, miRNAs are expressed in a highly tissue-specific or developmentally regulated manner and this regulation is likely key to their predicted roles in eukaryotic development and differentiation. Analysis of the normal role of miRNAs will be facilitated by techniques that allow the regulated over-expression or inappropriate expression of authentic miRNAs in vivo, whereas the ability to regulate the expression of siRNAs will greatly increase their utility both in cultured cells and in vivo. Thus one can design and express artificial microRNAs based on the features of existing microRNA genes, such as the gene encoding the human miR-30 microRNA. These miR30-based shRNAs have complex folds, and, compared with simpler stem/loop style shRNAs, are more potent at inhibiting gene expression in transient assays.

miRNAs are first transcribed as part of a long, largely single-stranded primary transcript (Lee et al., EMBO J. 21: 4663-4670, 2002). This primary miRNA transcript is generally, and possibly invariably, synthesized by RNA polymerase II (pol II) and therefore is normally polyadenylated and may be spliced. It contains an 80-nt hairpin structure that encodes the mature ˜22-nt miRNA as part of one arm of the stem. In animal cells, this primary transcript is cleaved by a nuclear RNaseIII-type enzyme called Drosha (Lee et al., Nature 425: 415-419, 2003) to liberate a hairpin miRNA precursor, or pre-miRNA, of ˜65 nt, which is then exported to the cytoplasm by exportin-5 and the GTP-bound form of the Ran cofactor (Yi et al., Genes Dev. 17: 3011-3016, 2003). Once in the cytoplasm, the pre-miRNA is further processed by Dicer, another RNaseIII enzyme, to produce a duplex of ˜22 bp that is structurally identical to an siRNA duplex (Hutvagner et al., Science 293: 834-838, 2001). The binding of protein components of the RNA-induced silencing complex (RISC), or RISC cofactors, to the duplex results in incorporation of the mature, single-stranded miRNA into a RISC or RISC-like protein complex, whereas the other strand of the duplex is degraded (Bartel, Cell 116: 281-297, 2004).

The miR-30 architecture can be used to express miRNAs or siRNAs from pol II promoter-based expression plasmids. See also Zeng et al., Methods in Enzymology 392: 371-380, 2005 (incorporated herein by reference).

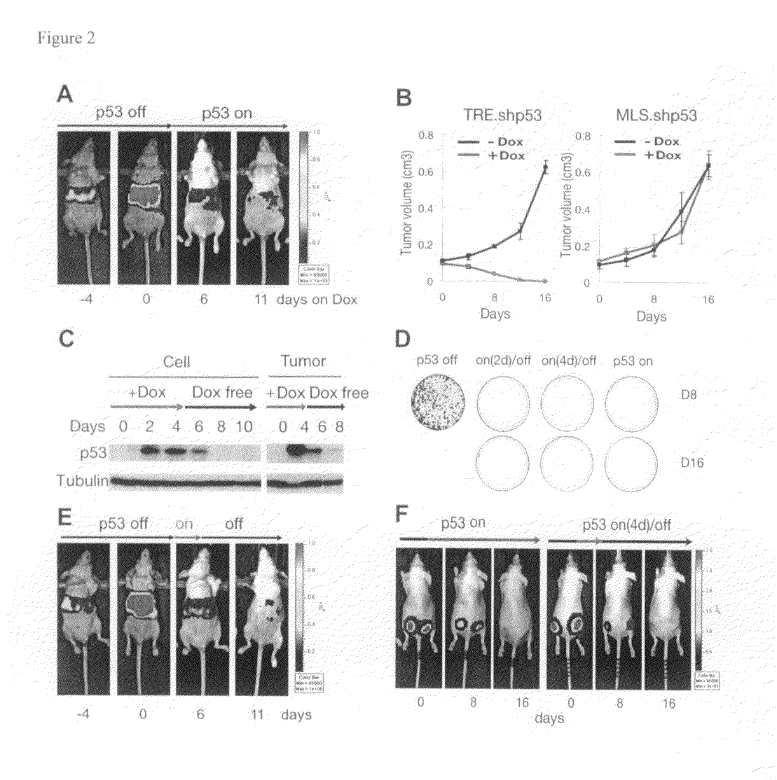

FIG. 2B of Zeng (supra) shows the predicted secondary structure of the miR-30 precursor hairpin (“the miR-30 cassette”). Boxed are extra nucleotides that were added originally for subcloning purposes (Zeng and Cullen, RNA 9: 112-123, 2003; Zeng et al., Mol. Cell. 9: 1327-1333, 2002). They represent XhoI-BglII sites at the 50 end and BamHI-XhoI sites at the 30 end. These appended nucleotides extend the minimal miR-30 precursor stem shown by several base pairs, similar to the in vivo situation where the primary miR-30 precursor is transcribed from its genomic locus (Lee et al., Nature 425: 415-419, 2003), and an extended stem of at least 5 bp is essential for efficient miR-30 production. Based on the numbering in FIG. 2B, mature miR-30 is encoded by nucleotides 44 to 65 and anti-miR-30 by nucleotides 3 to 25 of this precursor. In the simplest expression setting, the cytomegalovirus (CMV) immediate early enhancer/promoter may be used to transcribe the miR-30 cassette. The cassette is preceded by a leader sequence of approximately 100 nt and followed by approximately 170 nt before the polyadenylation site (Zeng et al., Mol. Cell. 9: 1327-1333, 2002). These lengths are arbitrary and can be longer or shorter. Mature 22-nt miR-30 can be made from such constructs.

Several other authentic miRNAs have been over-expressed by using analogous RNA pol II-based expression vectors or even pol III-dependent promoters (Chen et al., Science 303: 83-86, 2004; Zeng and Cullen, RNA 9: 112-123, 2003). Expression simply requires the insertion of the entire predicted miRNA precursor stem-loop structure into the expression vector at an arbitrary location. Because the actual extent of the precursor stem loop can sometimes be difficult to accurately predict, it is generally appropriate to include ˜50 bp of flanking sequence on each side of the predicted ˜80-nt miRNA stem-loop precursor to be sure that all cis-acting sequences necessary for accurate and efficient Drosha processing are included (Chen et al., Science 303: 83-86, 2004).

In an exemplary embodiment, to make the miR-30 expression cassette, the sequence from +1 to 65 (excluding the 15-nt terminal loop of the miR-30 cassette, FIG. 2B of Zeng) may be replaced as follows: the sequence from nucleotides 39 to 61, which is perfectly complementary to a target gene sequence, will act as the active strand during RNAi. The sequence from nucleotides 2 to 23 is thus designed to preserve the double-stranded stem in the miR-30-target cassette, but nucleotide +1 is now a C, to create a mismatch with nucleotide 61, a U, just like nucleotides 1 and 65 in the miR-30 cassette (FIG. 2B). Because the 3′ arm of the stem (miR-30-target) is the active component for RNAi, changes in the 5′ arm of the stem will not affect RNAi specificity. A 2-nt bulge may be present in the stem region of the authentic miR-30 precursor (FIG. 2B of Zeng). A break in the helical nature of the RNA stem may help ward off nonspecific effects, such as induction of an interferon response (Bridge et al., Nat. Genet. 34: 263-264, 2003) in expressing cells. This may be why miRNA precursors almost invariably contain bulges in the predicted stem. The miR-30 cassette in FIG. 2A of Zeng is then substituted with the miR-30-target cassette, and the resulting expression plasmid can be transfected into target cells.

The use of pol II promoters, especially when coupled with an inducible expression system (such as the TetOFF system of Clontech) offers flexibility in regulating the production of miRNAs in cultured cells or in vivo. Selection of stable cell lines leads to less leaky expression in the absence of the activator or presence of doxycycline, and therefore a stronger induction.

In certain embodiments, it would be advantageous if the antisense strand, for example, of the above miR-30-target construct is preferentially made as a mature miRNA, because its opposite strand does not have any known target. The relative base pairing stability at the 5′ ends of an siRNA duplex is a strong determinant of which strand will be incorporated into RISC and hence be active in RNAi; the strand whose 5′ end has a weaker hydrogen bonding pattern is preferentially incorporated into RISC, the RNAi effecter complex (Khvorova et al., Cell 115: 209-216, 2003; Schwarz et al., Cell 115: 208-299, 2003). This same principle can also be applied to the design of DNA vector-based siRNA expression strategies, including the one described here. However, for artificial miRNAs, the fact that the internal cleavage sites by Drosha and Dicer cannot be precisely predicted at present adds a degree of uncertainty as a 1- or 2-nt shift in the cleavage site can generate rather different hydrogen bonding patterns at the 50 ends of the resulting duplex, thus changing which strand of the duplex intermediate is incorporated into RISC. This is in contrast to the situation with synthetic siRNA duplexes, which have defined ends. On the other hand, any minor heterogeneity at the ends of an artificial miRNA duplex intermediate might not be a problem, as the miRNAs would still be perfectly complementary to their target.

The role of internal loop, stem length, and the surrounding sequences on the expression of miRNAs from miR-30-derived cassettes may also be systematically examined to optimize expression of the miR-based shRNA. Such analyses may suggest design elements that would maximize the yield of the intended RNA products. On the other hand, some heterogeneity could be inevitable. In addition to the 5′-end rule, specific residues at some positions within an siRNA may also enhance siRNA function (Reynolds et al., Nat. Biotech. 22: 326-330, 2004).

In general, picking a target region with more than 50% AU content and designing a weak 50 end base pair on the antisense strand would be a good starting point in the design of any artificial miRNA/siRNA expression plasmid (Khvorova et al., Cell 115: 209-216, 2003; Reynolds et al., Nat. Biotech. 22: 326-330, 2004; Schwarz et al., Cell 115: 208-299, 2003).

In certain embodiments, expression of the miR-30 cassette may be in the antisense orientation, especially when the cassette is to be used in lentiviral or retroviral vectors. This is partly because miRNA processing may result in the degradation of the remainder of the primary miRNA transcript.

In other embodiments, vectors may contain inserts expressing more than one miRNAs. In such constructs, the fact that each miRNA stem-loop precursor is independently excised from the primary transcript by Drosha cleavage to give rise to a pre-miRNA allows simultaneous expression of several artificial or authentic miRNAs by a tandem array on a precursor RNA transcript.

Genome wide libraries of shRNAs based on the miR30 precursor RNA have also been generated. Each member of such libraries target specific human or mouse genes, and may be readily converted to the vectors/expression systems of the instant invention. The following section describes the design of such libraries.

Silva et al. (Nature Genetics 37(11): 1281-8, 2005); Dickins et al. (Nature Genetics 37: 1289, 2005), and Stegmeier et al. (Proc Natl Acad Sci U.S.A. 102(37): 13212-7, 2005), all incorporated herein by reference, have described a genome-wise library of shRNAs based on the miR30 precursor RNA, which may be adapted for use in the instant invention. The described vector pSHAG-MAGIC2 (pSM2) is roughly equivalent to pSHAG-MAGIC1 as described in Paddison et al. Methods Mol. Biol. 265: 85-100 (2004), incorporated herein by reference. The few notable exceptions include: the new cloning strategy is based on the use of a single oligonucleotide that contains the hairpin and common 5′ and 3′ ends as a PCR template (see FIG. 2 of Paddison, Nature Methods 1(2): 163-67, 2004). The resulting PCR product is then cloned into the hairpin cloning site of the pSM2 vector, which drives miR-30-styled hairpins by the human U6 promoter. Inserts from this library may be excised (see Example below) and cloned into the instant vectors for Pol II-driven expression of the same miR-30-styled hairpins. This allows the instant methods to be coupled with the existing library of miR-30-style constructs that contains most human and mouse genes.

Paddison also describes the detailed methods for designing 22-nucleotide sequences (targeting a target gene) that can be inserted into the precursor miRNA, PCR protocols for amplification, and relevant critical steps and trouble-shootings, etc. (all incorporated herein by reference).

MicroRNAs (including the siRNA products and artificial microRNAs as well as endogenous microRNAs) have potential for use as therapeutics as well as research tools, e.g. analyzing gene function. As a general method, the mature microRNA (miR) of the invention, especially those non-miR-30 based microRNA constructs of the invention may also be produced according to the following description.

In certain embodiments, the methods for efficient expression of microRNA involve the use of a precursor microRNA molecule having a microRNA sequence in the context of microRNA flanking sequences. The precursor microRNA is composed of any type of nucleic acid based molecule capable of accommodating the microRNA flanking sequences and the microRNA sequence. Examples of precursor microRNAs and the individual components of the precursor (flanking sequences and microRNA sequence) are provided herein. The invention, however, is not limited to the examples provided. The invention is based, at least in part, on the discovery of an important component of precursor microRNAs, that is, the microRNA flanking sequences. The nucleotide sequence of the precursor and its components may vary widely.

In one aspect a precursor microRNA molecule is an isolated nucleic acid including microRNA flanking sequences and having a stem-loop structure with a microRNA sequence incorporated therein. An “isolated molecule” is a molecule that is free of other substances with which it is ordinarily found in nature or in vivo systems to an extent practical and appropriate for its intended use. In particular, the molecular species are sufficiently free from other biological constituents of host cells or if they are expressed in host cells they are free of the form or context in which they are ordinarily found in nature. For instance, a nucleic acid encoding a precursor microRNA having homologous microRNA sequences and flanking sequences may ordinarily be found in a host cell in the context of the host cell genomic DNA. An isolated nucleic acid encoding a microRNA precursor may be delivered to a host cell, but is not found in the same context of the host genomic DNA as the natural system. Alternatively, an isolated nucleic acid is removed from the host cell or present in a host cell that does not ordinarily have such a nucleic acid sequence. Because an isolated molecular species of the invention may be admixed with a pharmaceutically-acceptable carrier in a pharmaceutical preparation or delivered to a host cell, the molecular species may comprise only a small percentage by weight of the preparation or cell. The molecular species is nonetheless isolated in that it has been substantially separated from the substances with which it may be associated in living systems.

An “isolated precursor microRNA molecule” is one which is produced from a vector having a nucleic acid encoding the precursor microRNA. Thus, the precursor microRNA produced from the vector may be in a host cell or removed from a host cell. The isolated precursor microRNA may be found within a host cell that is capable of expressing the same precursor. It is nonetheless isolated in that it is produced from a vector and, thus, is present in the cell in a greater amount than would ordinarily be expressed in such a cell.

The term “nucleic acid” is used to mean multiple nucleotides (i.e. molecules comprising a sugar (e.g. ribose or deoxyribose) linked to a phosphate group and to an exchangeable organic base, which is either a substituted pyrimidine (e.g. cytosine (C), thymidine (T) or uracil (U)) or a substituted purine (e.g. adenine (A) or guanine (G)). The term shall also include polynucleosides (i.e. a polynucleotide minus the phosphate) and any other organic base containing polymer. Purines and pyrimidines include but are not limited to adenine, cytosine, guanine, thymidine, inosine, 5-methylcytosine, 2-aminopurine, 2-amino-6-chloropurine, 2,6-diaminopurine, hypoxanthine, and other naturally and non-naturally occurring nucleobases, substituted and unsubstituted aromatic moieties. Other such modifications are well known to those of skill in the art. Thus, the term nucleic acid also encompasses nucleic acids with substitutions or modifications, such as in the bases and/or sugars.

“MicroRNA flanking sequence” as used herein refers to nucleotide sequences including microRNA processing elements. MicroRNA processing elements are the minimal nucleic acid sequences which contribute to the production of mature microRNA from precursor microRNA. Often these elements are located within a 40 nucleotide sequence that flanks a microRNA stem-loop structure. In some instances the microRNA processing elements are found within a stretch of nucleotide sequences of between 5 and 4,000 nucleotides in length that flank a microRNA stem-loop structure.

Thus, in some embodiments the flanking sequences are 5-4,000 nucleotides in length. As a result, the length of the precursor molecule may be, in some instances at least about 150 nucleotides or 270 nucleotides in length. The total length of the precursor molecule, however, may be greater or less than these values. In other embodiments the minimal length of the microRNA flanking sequence is 10, 20, 30, 40, 50, 60, 70, 80, 90, 100, 150, 200 and any integer there between. In other embodiments the maximal length of the microRNA flanking sequence is 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900 4,000 and any integer there between.

The microRNA flanking sequences may be native microRNA flanking sequences or artificial microRNA flanking sequences. A native microRNA flanking sequence is a nucleotide sequence that is ordinarily associated in naturally existing systems with microRNA sequences, i.e., these sequences are found within the genomic sequences surrounding the minimal microRNA hairpin in vivo. Artificial microRNA flanking sequences are nucleotides sequences that are not found to be flanking to microRNA sequences in naturally existing systems. The artificial microRNA flanking sequences may be flanking sequences found naturally in the context of other microRNA sequences. Alternatively they may be composed of minimal microRNA processing elements which are found within naturally occurring flanking sequences and inserted into other random nucleic acid sequences that do not naturally occur as flanking sequences or only partially occur as natural flanking sequences.

The microRNA flanking sequences within the precursor microRNA molecule may flank one or both sides of the stem-loop structure encompassing the microRNA sequence. Thus, one end (i.e., 5′) of the stem-loop structure may be adjacent to a single flanking sequence and the other end (i.e., 3′) of the stem-loop structure may not be adjacent to a flanking sequence. Preferred structures have flanking sequences on both ends of the stem-loop structure. The flanking sequences may be directly adjacent to one or both ends of the stem-loop structure or may be connected to the stem-loop structure through a linker, additional nucleotides or other molecules.

A “stem-loop structure” refers to a nucleic acid having a secondary structure that includes a region of nucleotides which are known or predicted to form a double strand (stem portion) that is linked on one side by a region of predominantly single-stranded nucleotides (loop portion). The terms “hairpin” and “fold-back” structures are also used herein to refer to stem-loop structures. Such structures are well known in the art and the term is used consistently with its known meaning in the art. The actual primary sequence of nucleotides within the stem-loop structure is not critical to the practice of the invention as long as the secondary structure is present. As is known in the art, the secondary structure does not require exact base-pairing. Thus, the stem may include one or more base mismatches. Alternatively, the base-pairing may be exact, i.e. not include any mismatches.

In some instances the precursor microRNA molecule may include more than one stem-loop structure. The multiple stem-loop structures may be linked to one another through a linker, such as, for example, a nucleic acid linker or by a microRNA flanking sequence or other molecule or some combination thereof.

In an alternative embodiment, useful interfering RNAs can be designed with a number of software programs, e.g., the OligoEngine siRNA design tool available at wwv.olioengine.com. The siRNAs of this invention may range about, e.g., 19-29 base pairs in length for the double-stranded portion. In some embodiments, the siRNAs are hairpin RNAs having an about 19-29 bp stem and an about 4-34 nucleotide loop. Preferred siRNAs are highly specific for a region of the target gene and may comprise any about 19-29 bp fragment of a target gene mRNA that has at least one, preferably at least two or three, bp mismatch with a no target gene-related sequence. In some embodiments, the preferred siRNAs do not bind to RNAs having more than 3 mismatches with the target region.

As described above, various vectors may be used to transduce into and express in host cells the antagonists (e.g., the siRNA constructs) to the tumor suppressor genes. The following section provides further details regarding several exemplary vectors and their uses. Other suitable vectors or variants may also be used in the instant invention.

The invention uses various vectors for producing precursor microRNA molecules. Generally these vectors include a sequence encoding a precursor microRNA and (in vivo) expression elements. The expression elements include at least one promoter, such as a Pol II promoter, which may direct the expression of the operably linked microRNA precursor (e.g. the shRNA encoding sequence). The vector or primary transcript is first processed to produce the stem-loop precursor molecule. The stem-loop precursor is then processed to produce the mature microRNA.

RNA polymerase III (Pol III) transcription units normally encode the small nuclear RNA U6 (see Tran et al., BMC Biotechnology 3: 21, 2003, incorporate herein by reference), or the human RNAse P RNA Hi. However, RNA polymerase II (Pol II) transcription units (e.g., units containing a modified minimal CMV promoter with Tet Responsive Elements, or “TRE-CMV”) is preferred for use with inducible expression. It will be appreciated that in the vectors of the invention, the subject shRNA encoding sequence may be operably linked to a variety of other promoters.

In some embodiments, the promoter is a type II tRNA promoter such as the tRNAVa promoter and the tRNAmet promoter. These promoters may also be modified to increase promoter activity. In addition, enhancers can be placed near the promoter to enhance promoter activity. Pol II enhancer may also be used for Pol III promoters. For example, an enhancer from the CMV promoter can be placed near the U6 promoter to enhance U6 promoter activity (Xia et al., Nuc Acids Res 31, 2003).

In certain embodiments, the subject Pol II promoters are inducible promoters. Exemplary inducible Pol II systems are available from Invitrogen, e.g., the GeneSwitch™ or T-REx™ systems; from Clontech (Palo Alto, Calif.), e.g., the TetON and TetOFF systems.

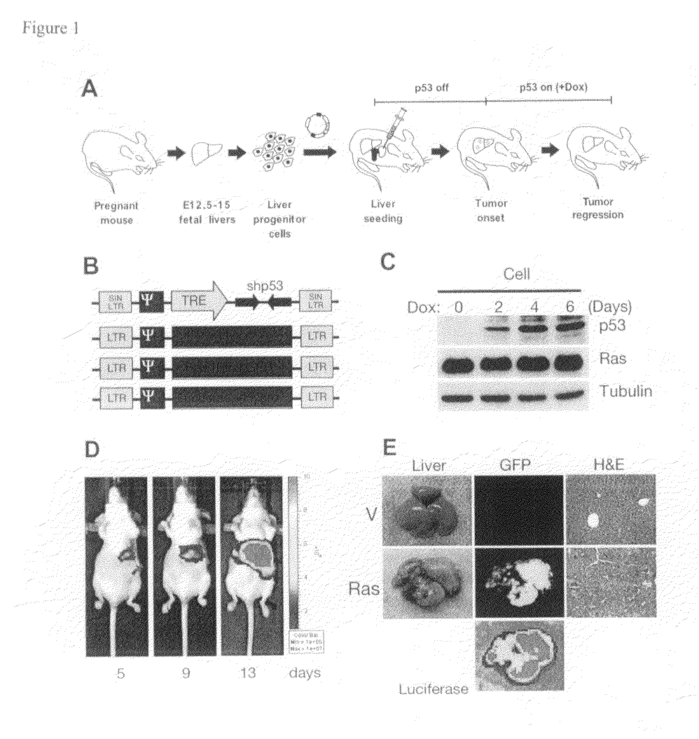

An exemplary Tet-responsive promoter is described in WO 04/056964 A2 (incorporated herein by reference). See, for example, FIG. 1 of WO 04/056964 A2. In one construct, a Tet operator sequence (TetOp) is inserted into the promoter region of the vector. TetOp is preferably inserted between the PSE and the transcription initiation site, upstream or downstream from the TATA box. In some embodiments, the TetOp is immediately adjacent to the TATA box. The expression of the subject shRNA encoding sequence is thus under the control of tetracycline (or its derivative doxycycline, or any other tetracycline analogue). Addition of tetracycline or Dox relieves repression of the promoter by a tetracycline repressor that the host cells are also engineered to express.

In the TetOFF system, a different tet transactivator protein is expressed in the tetOFF host cell. The difference is that Tet/Dox, when bind to an activator protein, is now capable to turn off transcriptional activation. Thus such host cells expressing the activator will only activate the transcription of an shRNA encoding sequence from a TetOFF promoter in the absence of Tet or Dox.

An alternative inducible promoter is a lac operator system, as illustrated in FIG. 2A of WO 04/056964 A2 (incorporated by reference). Briefly, a Lac operator sequence (LacO) is inserted into the promoter region. The LacO is preferably inserted between the PSE and the transcription initiation site, upstream or downstream of the TATA box. In some embodiments, the LacO is immediately adjacent to the TATA box. The expression of the RNAi molecule (shRNA encoding sequence) is thus under the control of IPTG (or any analogue thereof). Addition of IPTG relieves repression of the promoter by a Lac repressor (i.e., the LacI protein) that the host cells are also engineered to express. Since the Lac repressor is derived from bacteria, its coding sequence may be optionally modified to adapt to the codon usage by mammalian transcriptional systems and to prevent methylation. In some embodiments, the host cells comprise (i) a first expression construct containing a gene encoding a Lac repressor operably linked to a first promoter, such as any tissue or cell type specific promoter or any general promoter, and (ii) a second expression construct containing the dsRNA-coding sequence operably linked to a second promoter that is regulated by the Lac repressor and IPTG. Administration of IPTG results in expression of dsRNA in a manner dictated by the tissue specificity of the first promoter.

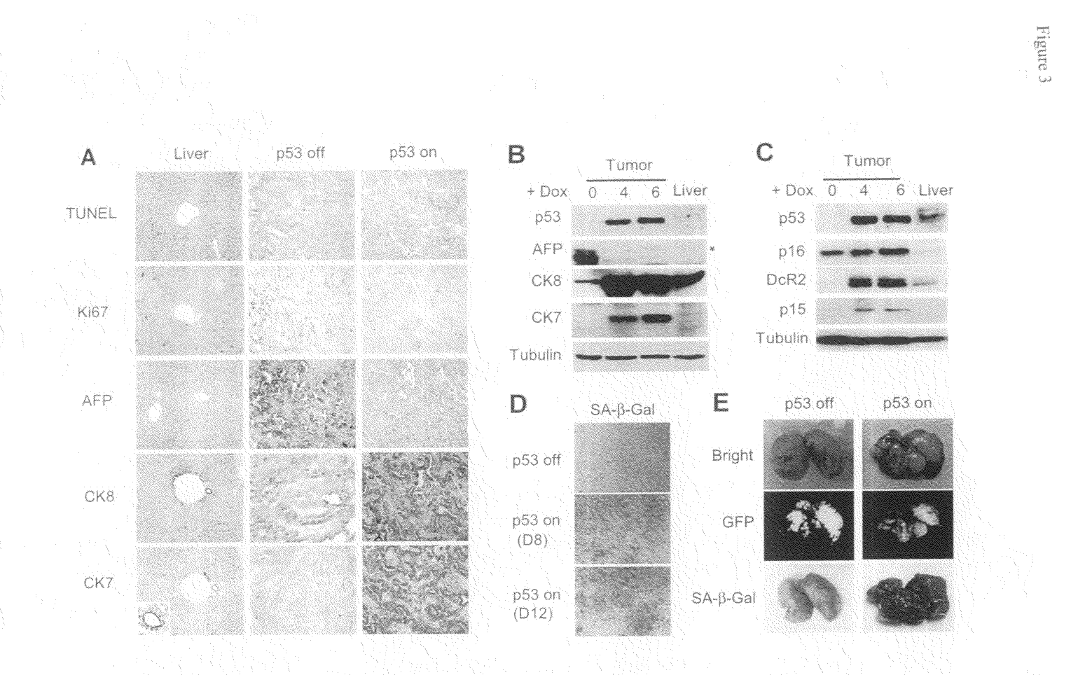

Yet another inducible system, a LoxP-stop-LoxP system, is illustrated in FIGS. 3A-3E of WO 04/056964 A2 (incorporated by reference). The RNAi vector of this system contains a LoxP-Stop-LoxP cassette before the hairpin or within the loop of the hairpin. Any suitable stop sequence for the promoter can be used in the cassette. One version of the LoxP Stop-LoxP system for Pol II is described in, e.g., Wagner et al., Nucleic Acids Research 25:4323-4330, 1997. The “Stop” sequences (such as the one described in Wagner, sierra, or a run of five or more T nucleotides) in the cassette prevent the RNA polymerase III from extending an RNA transcript beyond the cassette. Upon introduction of a Cre recombinase, however, the LoxP sites in the cassette recombine, removing the Stop sequences and leaving a single LoxP site. Removal of the Stop sequences allows transcription to proceed through the hairpin sequence, producing a transcript that can be efficiently processed into an open-ended, interfering dsRNA. Thus, expression of the RNAi molecule is induced by addition of Cre.

In some embodiments, the host cells contain a Cre-encoding transgene under the control of a constitutive, tissue-specific promoter. As a result, the interfering RNA can only be inducibly expressed in a tissue-specific manner dictated by that promoter. Tissue-specific promoters that can be used include, without limitation: a tyrosinase promoter or a TRP2 promoter in the case of melanoma cells and melanocytes; an MMTV or WAP promoter in the case of breast cells and/or cancers; a Villin or FABP promoter in the case of intestinal cells and/or cancers; a RIP promoter in the case of pancreatic beta cells; a Keratin promoter in the case of keratinocytes; a Probasin promoter in the case of prostatic epithelium; a Nestin or GFAP promoter in the case of CNS cells and/or cancers; a Tyrosine Hydroxylase, S1100 promoter or neurofilament promoter in the case of neurons; the pancreas-specific promoter described in Edlund et al., Science 230: 912-916, 1985; a Clara cell secretory protein promo-ter in the case of lung cancer; and an Alpha myosin promoter in the case of cardiac cells.

Cre expression also can be controlled in a temporal manner, e.g., by using an inducible promoter, or a promoter that is temporally restricted during development such as Pax3 or Protein O (neural crest), Hoxal (floorplate and notochord), Hoxb6 (extraembryonic mesoderm, lateral plate and limb mesoderm and midbrain-hindbrain junction), Nestin (neuronal lineage), GFAP (astrocyte lineage), Lck (immature thymocytes). Temporal control also can be achieved by using an inducible form of Cre. For example, one can use a small molecule controllable Cre fusion, for example a fusion of the Cre protein and the estrogen receptor (ER) or with the progesterone receptor (PR). Tamoxifen or RU486 allow the Cre-ER or Cre-PR fusion, respectively, to enter the nucleus and recombine the LoxP sites, removing the LoxP Stop cassette. Mutated versions of either receptor may also be used. For example, a mutant Cre-PR fusion protein may bind RU486 but not progesterone. Other exemplary Cre fusions are a fusion of the Cre protein and the glucocorticoid receptor (GR). Natural GR ligands include corticosterone, cortisol, and aldosterone. Mutant versions of the GR receptor, which respond to, e.g., dexamethasone, triamcinolone acetonide, and/or RU38486, may also be fused to the Cre protein.

In certain embodiments, additional transcription units may be present 3′ to the shRNA portion. For example, an internal ribosomal entry site (IRES) may be positioned downstream of the shRNA insert, the transcription of which is under the control of a second promoter, such as the PGK promoter. The IRES sequence may be used to direct the expression of a operably linked second gene, such as a reporter gene. The reporter gene may be a fluorescent protein, such as GFP, RFP, BFP, YFP, etc., an enzyme such as luciferase (Promega), etc., or any other art-recognized reporter whose physical presence and/or activity can be readily assessed using an art-recognized method. The reporter gene may serve as an indication of infection/transfection, and the efficiency and/or amount of mRNA transcription of the shRNA—IRES—reporter cassette/insert. Optionally, one or more selectable markers (such as puromycin resistance gene, neomycin resistance gene, hygromycin resistance gene, zeocin resistance gene, etc.) may also be present on the same vector, and are under the transcriptional control of the second promoter. Such markers may be useful for selecting stable integration of the vector into a host cell genome.

Certain variant vectors may also be used for the invention. In general, variants typically will share at least 40% nucleotide identity with any of the described vectors, in some instances, will share at least 50% nucleotide identity; and in still other instances, will share at least 60% nucleotide identity. The preferred variants have at least 70% sequence homology. More preferably the preferred variants have at least 80% and, most preferably, at least 90% sequence homology to the described sequences.

Variants with high percentage sequence homology can be identified, for example, using stringent hybridization conditions. The term “stringent conditions”, as used herein, refers to parameters with which the art is familiar. More specifically, stringent conditions, as used herein, refer to hybridization at 65° C. in hybridization buffer (3.5×SSC, 0.02% Ficoll, 0.02% polyvinyl pyrolidone, 0.02% bovine serum albumin, 2.5 mM NaH₂PO₄ (pH 7), 0.5% SDS, 2 mM EDTA). SSC is 0.15M sodium chloride/0.15M sodium citrate, pH 7; SDS is sodium dodecyl sulphate; and EDTA is ethylenediaminetetraacetic acid. After hybridization, the membrane to which the DNA is transferred is washed at 2×SSC at room temperature and then at 0.1×SSC/0.1×SDS at 65° C. There are other conditions, reagents, and so forth which can be used, which result in a similar degree of stringency. Such variants may be further subject to functional testing such that variants that substantially preserve the desired/relevant function of the original vectors are selected/identified.

The “in vivo expression elements” are any regulatory nucleotide sequence, such as a promoter sequence or promoter-enhancer combination, which facilitates the efficient expression of the nucleic acid to produce the precursor microRNA. The in vivo expression element may, for example, be a mammalian or viral promoter, such as a constitutive or inducible promoter or a tissue specific promoter. Constitutive mammalian promoters include, but are not limited to, polymerase II promoters as well as the promoters for the following genes: hypoxanthine phosphoribosyl transferase (HPTR), adenosine deaminase, pyruvate kinase, and β-actin. Exemplary viral promoters which function constitutively in eukaryotic cells include, for example, promoters from the simian virus, papilloma virus, adenovirus, human immunodeficiency virus (HIV), Rous sarcoma virus, cytomegalovirus, the long terminal repeats (LTR) of moloney leukemia virus and other retroviruses, and the thymidine kinase promoter of herpes simplex virus. Other constitutive promoters are known to those of ordinary skill in the art. The promoters useful as in vivo expression element of the invention also include inducible promoters. Inducible promoters are expressed in the presence of an inducing agent. For example, the metallothionein promoter is induced to promote transcription in the presence of certain metal ions. Other inducible promoters are known to those of ordinary skill in the art.

One useful inducible expression system that can be adapted for use in the instant invention is the Tet-responsive system, including both the TetON and TetOFF embodiments.

TetOn system is a commercially available inducible expression system from Clontech Inc. This is of particular interest because current siRNA expression systems utilize pol III promoters, which are difficult to adapt for inducible expression. The Clontech TetON system includes the pRev-TRE vector, which can be packaged into retrovirus and used to infect a Tet-On cell line expressing the reverse tetracycline-controlled transactivator (rtTA). Once introduced into the TetON host cell, the shRNA insert can then be inducibly expressed in response to varying concentrations of the tetracycline derivate doxycycline (Dox).

In general, the in vivo expression element shall include, as necessary, 5′ non-transcribing and 5′ non-translating sequences involved with the initiation of transcription. They optionally include enhancer sequences or upstream activator sequences as desired.

Vectors include, but are not limited to, plasmids, phagemids, viruses, other vehicles derived from viral or bacterial sources that have been manipulated by the insertion or incorporation of the nucleic acid sequences for producing the precursor microRNA, and free nucleic acid fragments which can be attached to these nucleic acid sequences. Viral and retroviral vectors are a preferred type of vector and include, but are not limited to, nucleic acid sequences from the following viruses: retroviruses, such as: Moloney murine leukemia virus; Murine stem cell virus, Harvey murine sarcoma virus; murine mammary tumor virus; Rous sarcoma virus; adenovirus; adeno-associated virus; SV40-type viruses; polyoma viruses; Epstein-Barr viruses; papilloma viruses; herpes viruses; vaccinia viruses; polio viruses; lentiviruses; and RNA viruses such as any retrovirus. One can readily employ other unnamed vectors known in the art.

Viral vectors are generally based on non-cytopathic eukaryotic viruses in which non-essential genes have been replaced with the nucleic acid sequence of interest. Non-cytopathic viruses include retroviruses, the life cycle of which involves reverse transcription of genomic viral RNA into DNA with subsequent proviral integration into host cellular DNA. Retroviruses have been approved for human gene therapy trials. Genetically altered retroviral expression vectors have general utility for the high-efficiency transduction of nucleic acids in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, M., “Gene Transfer and Expression, A Laboratory Manual,” W.H. Freeman Co., New York (1990) and Murry, E. J. Ed. “Methods in Molecular Biology,” vol. 7, Humana Press, Inc., Cliffton, N.J. (1991).

Exemplary vectors are disclosed herein and in US 2005/0075492 A2 (incorporated herein by reference) and WO 04/056964 A2 (incorporated herein by reference).

The invention also encompasses host cells transfected with the subject vectors, especially host cell lines with stably integrated shRNA or microRNA constructs. In certain embodiments, the subject host cell contains only a single copy of the integrated construct expressing the desired shRNA or microRNA (optionally under the control of an inducible and/or tissue specific promoter). Host cells include for instance, cells (such as primary cells or embryonic progenitor cells) and cell lines.

The invention also encompasses animals comprising host cells transfected with the subject vectors, especially host cell lines with stably integrated shRNA or microRNA constructs. In certain embodiments, the subject animals may comprise a germline transgene capable of expressing a subject oncogene, siRNA construct targeting a subject tumor suppressor gene, or a subject marker gene. The transgene may be iniquitously expressed, or only expressed in a tissue-specific or developmental stage-specific manner. The expression of the transgene may be inducible and/or reversible, or may be constitutive.

Although many different embodiments of the inventions are described above separately, in parallel, and/or in different sections, it is contemplated that any one embodiment may be combined with any other embodiments where appropriate.

### EXAMPLES

The invention now being generally described, it will be more readily understood by reference to the following examples, which are included merely for purposes of illustration of certain aspects and embodiments of the present invention, and are not intended to limit the invention.

### Example 1

Although cancer arises from a combination of mutations in oncogenes and tumor suppressor genes, the extent to which tumor suppressor gene loss is required for the maintenance of established tumors is poorly understood. Using a conditional RNA interference and a mosaic mouse model of liver carcinoma, Applicants demonstrate that even brief reactivation of endogenous p53 in p53-deficient tumors can produce complete tumor regressions in vivo. Applicants also made the surprising discovery that hepatocarcinomas did not display apoptosis in response to p53 reactivation. Instead, reactivated p53 activated a senescence program that was associated with cellular differentiation and the upregulation of inflammatory cytokines. This program, while producing only cell cycle arrest in vitro, also triggered an innate immune response that targeted the tumor cells and vasculature, thereby contributing to tumor clearance. Thus Applicants have demonstrated that p53 loss is required for the maintenance of aggressive carcinomas, and have identified a novel mechanism by which a cellular senescence program can act together with the innate immune system to potently limit tumor growth.

Mutations in the p53 tumor suppressor gene are often associated with aggressive tumor behavior and poor patient prognosis (1). In normal cells, p53 acts to restrict proliferation in response to DNA damage or deregulation of mitogenic oncogenes, leading to the induction of cell cycle checkpoints, apoptosis, and cellular senescence (2, 3). These processes provide a potent barrier to tumorigenesis, but from which the majority of human cancers eventually escape.

While enforced overexpression of exogenous p53 can lead to the arrest or death of malignant tumor cells (5), the consequences of restoring the endogenous p53 pathway in tumors are unknown. Indeed, since p53 loss comprises cell cycle checkpoints that maintain genome integrity, it is quite possible that genomic instability may have driven tumor cells beyond their dependence on p53 mutations.

One tumor type where p53 mutations are common is human liver cancer (6), which is typically highly aggressive and resistant to non-surgical therapies. To address the requirement of p53 loss for the maintenance of such carcinomas, Applicants used reversible RNA interference (7) in a chimeric mouse model, where liver carcinomas are produced by ex vivo genetic manipulation of hepatocytes, followed by their retransplantation into recipient mice (FIG. 1A) (8, 9).

Specifically, isolation, culture and retroviral infection of murine hepatoblasts were described recently (8, 9). Purified embryonic liver progenitor cells (hepatoblasts) were transduced with MCSV retroviruses expressing oncogenic ras (H-ras-V12), the tetracycline transactivator protein tTA (“tet-off” system) and a miR30-design shRNA against murine p53 (shp53) driven by the TRE-CMV promoter (FIG. 1B) (7). To facilitate in vivo imaging, the oncogenic ras allele co-expressed green fluorescent protein and, in some experiments, hepatoblasts were also co-transduced with a luciferase reporter (selected with hygromycin).

The luciferase-hygro vector was generated by cloning the luciferase cDNA (pGL3, Promega) into the MSCV-Hygro vector (Clontech). Generation of all other vectors has been described recently (7).

Genetically modified hepatoblasts were introduced into the livers of retrorsine pretreated (8) female NCR nu/nu mice (6-8 weeks of age) by intra-splenic injection. Transplanted cells were allowed to migrate to the recipient liver and engraft the organ. Tumor progression or regression was monitored by abdominal palpation, whole body GFP imaging and in vivo bioluminescence imaging. To generate subcutaneous tumors, female nude mice (NCR nu/nu) were γ-irradiated (400 rad) and 3×10⁶ cells (unless otherwise noted in the figure legend) were subcutaneously injected into the rear flanks of the mice. Tumor volume (cm³) was determined by caliper measurement and calculated as 0.52×length×width×width.

Doxycycline (BD) was refreshed in cell culture medium (100 ng/mL) every 2 days. Mice were treated with 0.2 mg/mL Dox in 0.5% sucrose solution in light-protected bottles. Dox was refreshed every 4 days. Bioluminescence imaging was performed on anaesthetized animals using a Xenogen imager. 200 μL luciferin salt (Xenogen, 15 mg/mL in PBS) was injected into mice (i.p.) 10-15 minutes before imaging. Exposure time was 30 seconds for animals and 10 seconds for explanted livers.

As expected, p53 expression was efficiently suppressed in the absence of Doxycycline (Dox, a tetracycline analog), and rapidly restored upon Dox addition (FIG. 1C). Upon transplantation into the livers of conditioned recipient mice, hepatoblast populations co-expressing ras and the conditional p53 shRNA rapidly produced invasive hepatocarcinomas in the absence of Dox (FIGS. 1D & 1E), whereas cells expressing vectors alone did not (data not shown). These tumors were GFP-positive, and, if produced using a luciferase reporter, could be visualized externally by bioluminescence imaging following administration of luciferin salt (FIGS. 1D & 1E). Consistent with their cell of origin, these tumors displayed histopathologies of human hepatocellular and cholangiocellular carcinoma (FIG. 1E).

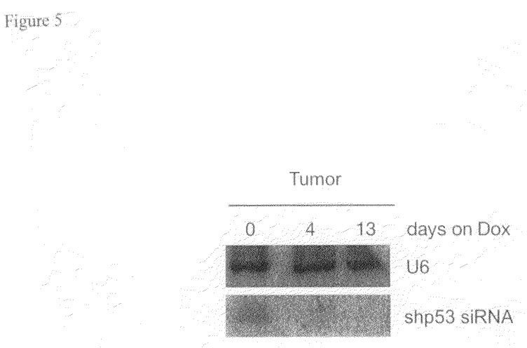

Upon the establishment of advanced tumors (FIGS. 1E & 2A), some animals were treated with Dox to turn off the p53 shRNA and re-establish p53 expression. Shortly after Dox administration, the processed p53 microRNA was efficiently shut off (FIG. 5), which correlated with an increase in p53 protein expression in vivo (see FIG. 2C). While tumors in untreated mice rapidly progressed (FIG. 1D), those in Dox-treated animals began to involute shortly after Dox administration, leading to nearly undetectable tumors within 12 days (FIG. 2A). Similar results were observed when the progenitor cells were transplanted subcutaneously into immunocompromised animals, where they could be accurately monitored using caliper measurements (FIG. 2B). Importantly, ras-induced liver carcinomas produced using a non-regulatable shRNA against p53 showed similar growth rates in the presence or absence of Dox (FIG. 2B, right panel), indicating that the tumor regressions observed were not simply due to Dox toxicity.

Such striking tumor regressions were not unique to tumors induced by oncogenic ras, but also occurred when p53 was reactivated in tumors co-expressing a constitutively activated Akt and the conditional p53 shRNA (data not shown).

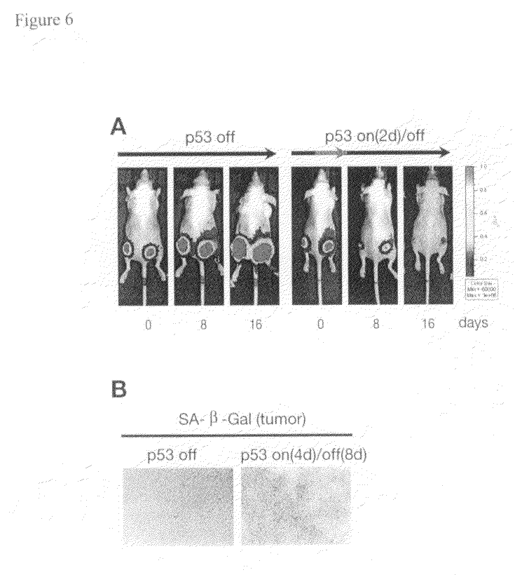

Previous work indicates that brief inactivation of the myc oncogene can induce the sustained regression of osteosarcomas in transgenic mice (10). To determine whether transient p53 reactivation can mimic chronic p53 action at inducing complete tumor remissions in our system, Applicants treated transformed cells in culture or tumor-bearing mice with Dox for 4 days, and then removed the drug. As shown by immunoblotting, the increase in p53 levels that followed Dox addition could be quickly reversed by Dox withdrawal (FIG. 2C). In cultured cells, even 2 days of Dox treatment was sufficient to reduce colony formation to levels observed in the continued presence of Dox (FIG. 2D). Furthermore, both in situ and subcutaneous liver carcinomas displayed complete regressions, similar to those observed following chronic p53 reactivation, after only 2-4 days of Dox treatment (FIGS. 2E, 2F, and 6A). Together, these data demonstrate that p53 loss is required for the maintenance and progression of aggressive carcinomas, and that p53 can induce tumor involution through a process that, once activated, appears irreversible.

The rapid involution of hepatocarcinomas re-expressing p53 is consistent with p53's well-characterized ability to promote apoptosis—a prominent form of tumor cell death that acts to limit tumor progression and can mediate the effects of some anticancer drugs (11). To gain insight into the mechanism of p53 induced tumor regression, Applicants next examined apoptosis and proliferation in tumors before and after p53 restoration by TUNEL and Ki-67 staining, respectively (FIG. 3A).

Surprisingly, based on these analysis, Applicants found that p53 did not induce apoptosis in tumor cells, at least at early time points when the tumors had begun to regress. Instead, these tumors displayed a marked decrease in proliferation that was associated with signs of cellular differentiation, including decreased expression of the embryonic liver- and liver tumor marker alpha-fetoprotein (AFP) and increased expression of the differentiation markers cytokeratin 8 (CK8) and cytokeratin 7 (CK7) (FIGS. 3A and 3B).

Hepatocarcinomas expressing either oncogenic ras or Akt displayed clear signs of senescence following p53 reactivation in vivo (FIGS. 3C-3E, data not shown). These included the accumulation of the established senescence markers p16^{INK4a}, DcR2, p15^{INK4b} (FIG. 3C) (13, 16), as well as the presence of senescence-associated-β-galactosidase (SA-β-Gal) activity (FIGS. 3D & 3E). SA-β-gal activity of comparable intensity was also observed in tumors following brief Dox treatment (FIG. 6B), indicating that a pulse of p53 activity was sufficient to trigger a senescence response in vivo.

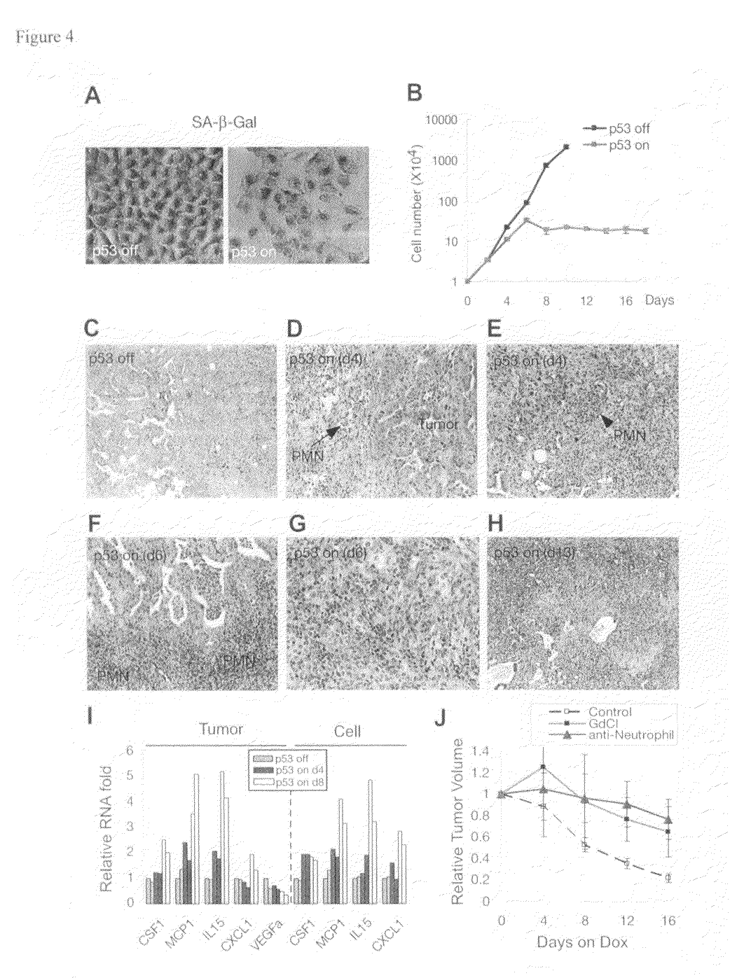

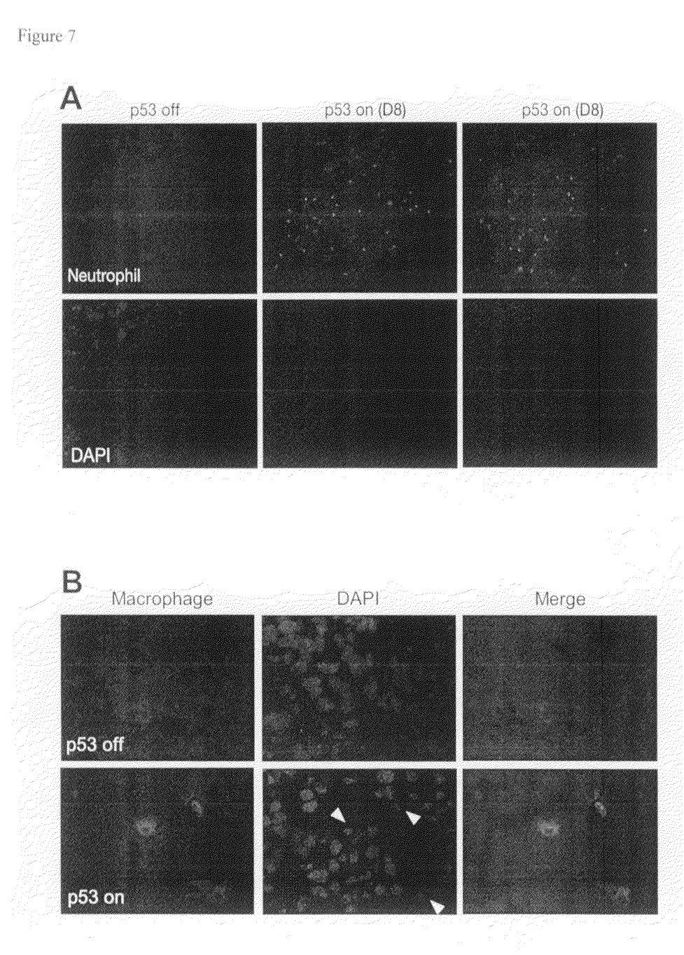

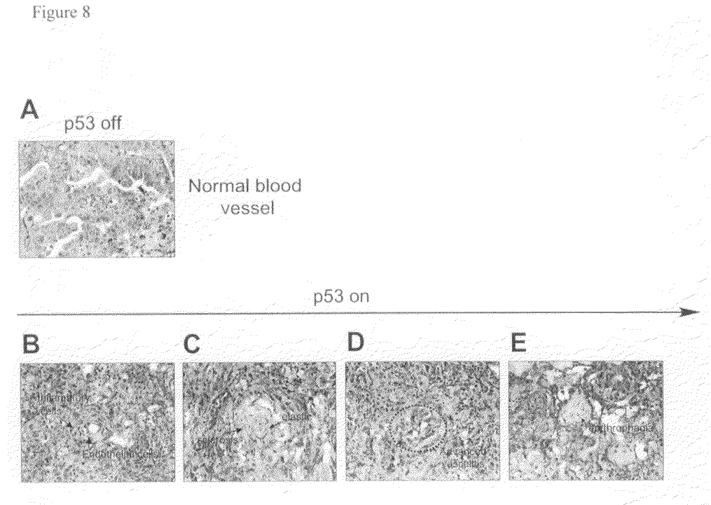

That p53 activation can induce both tumor cell senescence and tumor involution is surprising given the cytostatic nature of the senescence program. Indeed, transformed cells triggered to undergo p53 reactivation in vitro accumulated SA-β-gal activity but remained arrested subsequently (FIGS. 4A & 4B), implying that tumor regression involves non-cell autonomous processes. In this regard, microscopic examination of a series of tumors harvested at different times following p53 reactivation revealed a progressive increase in inflammatory infiltrates in the tumor. Although no overt immune response was noted in untreated carcinomas (FIG. 4C) or those 2 days after Dox treatment (data not shown), within 4 days an inflammatory reaction composed mainly of polymorphonuclear leukocytes (PMNs) developed that initially was most pronounced in peri-tumoral regions (FIG. 4D). At later times, this PMN reaction expanded to both involve intratumoral infiltration (FIGS. 4E-4H) and perivascular foci (FIG. 8B). Immunofluorescence analysis on tumor sections confirmed that neutrophil granulocytes and macrophages were major components of the immune infiltrate (FIG. 7). At day 6, the PMNs had spread throughout the tumor (FIG. 4F), forming cellular reach foci (FIG. 4G). Day 13 after p53 reactivation, the tumor architecture was largely damaged by the infiltrating leukocytes (FIG. 4H).

In the regressing tumor, Applicants also observed more intense perivascular infiltration, characterized by ‘plumbed’ (enlarged) endothelial cells and distorted lumens, damaging mainly mid-size blood vessels (FIG. 8B). By day 8, and more obvious at day 13, Applicants observed an overt vasculitis, producing sclerosed vessels, hemorraghia and erythrophagocytosis (FIGS. 8C-8E). These histopathological features support a model of sequential events, initiated by p53 reactivation in the tumor, activation of a dramatic inflammatory response, followed by destruction of tumor cells and neo-vasculature.

In addition to their permanent cell cycle arrest, another hallmark of cellular senescence is a dramatic change in gene expression that includes the upregulation of genes encoding inflammatory cytokines and other immune modulators (18-20). Applicants reasoned that such factors might recruit components of the immune system to the tumor mass, thereby assisting in the clearance of tumor cells. Consistent with this prediction, Applicants noted upregulation of several chemokines in the tumors following p53 reactivation, which are known to attract either macrophages (CSF1 and MCP1) or neutrophils (IL-15 and CXCL1) (FIG. 4I, left). While increased mRNA expression levels for these leukocyte attracting chemokines were already found 4 days after p53 restoration, even higher expression levels were detected later on. Importantly, these genes were also upregulated in transformed cells following p53 reactivation in culture, demonstrating that they are produced, at least in part, by the hepatoma cells rather than by infiltrating immune cells (FIG. 4I, right). Using expression profiling, we also noted an increase in transcripts corresponding to the angiogenesis inhibitors thrombospondin 2 (thbs2) and thrombospondin 4 (thbs4) following p53 activation (2.5- and 4-fold increase, respectively; data not shown). The increased secretion of such factors may contribute to the late stage vasculitis Applicants observed.

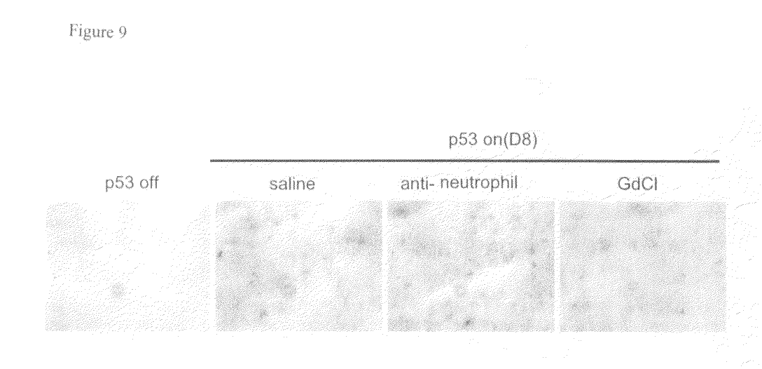

To determine whether components of the innate immune system were required for tumor cell clearance, mice harboring subcutaneous hepatocarcinomas co-expressing oncogenic ras and the conditional p53 shRNA were treated with gadolinium chloride (a macrophage toxin) or high doses of an anti-neutrophil antibody to suppress macrophages or neutrophils, respectively. Animals were then monitored for tumor regression following Dox administration. Both treatments significantly delayed tumor regression upon p53 reactivation, thus confirming that macrophages and neutrophils were actively involved in tumor clearance (FIG. 4J). Importantly, administration of gadolinium chloride or the anti-neutrophil antibody did not prevent tumor senescence as assessed by SA-β-gal activity (FIG. 9). These results indicate that the induction of cellular senescence and tumor attack by the innate immune system cooperate to promote tumor clearance.

In summary, Applicants used in vivo RNA interference technology to conditionally regulate endogenous p53 expression in vivo, and in doing so, demonstrated that p53 loss is required for the maintenance and progression of aggressive hepatocarcinomas. Thus, similar to certain oncogenes such as myc and ras, tumors can be “addicted” to p53 mutations and can not tolerate the restoration of normal p53 function.

Surprisingly, tumor cells here respond to p53 reactivation by undergoing a program of senescence, which has features of differentiation and triggers an innate immune response as well as disturbance of neo-vasculature. Although it is possible that some tumors may eventually escape their dependence on p53 mutations, the fact that brief reactivation caused complete tumor regressions in our system supports the use of transient p53 gene therapy approaches or small molecule drugs that reactivate mutant p53 or inhibit wild-type p53 turnover by mdm2 (24, 25), even for advanced cancers.

Results described herein also identify a novel mechanism of tumor suppression involving cooperative interactions between a tumor cell senescence program and the innate immune system and, as such, have important implications for cancer biology and therapy. First, they demonstrate that, despite the cytostatic nature of the program, senescent cells can turn over in vivo. Such cell clearance may reinforce the action of senescence as a barrier against tumorigenesis, as well as explain the ultimate regression of human tumors following senescence or differentiation promoting therapies (26-28). Second, they suggest that senescent tumor cells secrete factors that trigger a non-cell autonomous program of tumor regression. Our study suggests that some secreted factors—when produced by the tumor cells—can have anti-tumor effects. Finally, our results identify a setting in which the innate immune system is provoked to attack tumor cells and neo-vasculature, thereby facilitating their elimination. As many aggressive tumors, such as liver carcinomas, are completely refractory to non-surgical therapies, strategies that harness these responses represent a promising therapeutic approach.

### Example 2

Senescence is a fail-safe mechanism to prevent malignant tumor, in that senescence program controlled by p53 and p16^{INK4a} contributes to the outcome of chemotherapies. In addition, some differentiation-inducing therapies also activate senescence pathways in tumors.

Example 1 above have shown that reactivation of p53 in the liver cancer model leads to tumor regression by inducing senescence and an accompanied immune response. Specifically, Applicants have shown that macrophages (and neutrophiles) are involved in clearing the senescent tumor cells in vivo. It is possible that senescent cells secret pro-inflammatory chemokines and up-regulating immune receptors that can trigger immune attack. Therefore, Applicants have established an in vitro model system to study how immune cells recognize and attack senescent cells, and what genes are involved in the process.

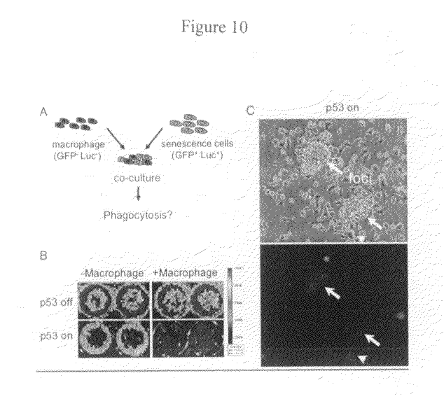

FIG. 10A is a schematic drawing showing the in vitro model system of the invention, comprising a co-culture of macrophages with senescent tumor cells following p53 reactivation. In this exemplary experiment, Ras;TRE.shp53;tTA liver tumor cells where generated, which liver tumor cells contains an activated Ras oncogene and a p53 shRNA-expressing construct under the control of tTA inducible promoter (see above). Upon Doxycycline treatment of the liver tumor cells for 4 days, p53 expression is turned on in the absence of the anti-p53 shRNA. The tumor cells with restored p53 expression were then co-cultured with mouse peritoneal macrophages. Tumor cells are shown in FIG. 10A as being positive for GFP and luciferase (Luc), while the macrophages are negative for both.

FIG. 10B shows a bioluminescence imaging of the co-culture. Duplicate wells are shown for each condition. It is apparent that, when p53 expression was turned off in the tumor cells, macrophages did not detectably engulf tumor cells via phagocytosis (compare the top row in FIG. 10B. However, after p53 expression was switched on and after the tumor cells went into senescence (bottom row), macrophages almost completely eliminated the bioluminent tumor cells.

FIG. 10C shows representative microscopic view of the co-culture. Arrows indicate senescent tumor cells (GFP positive) covered by GFP negative macrophages.

Therefore, these data demonstrated that co-culturing of macrophages with senescent tumor cells reduced tumor cell viability. This in vitro model/assay system provides an important platform to identify factors that may modulate (especially, enhance) the ability of the innate immune system (such as macrophages) to engulf senescent tumor cells, and to identify genes, marker, or receptors involved in this process.

Some data suggests that certain cell-surface adhesion molecules may be up-regulated by the senescence program. Without limitation, such adhesion molecules may include ICAM1, VCAM1, NCAM1, etc. While not wishing to be bound by any particular theory, these adhesion molecules expressed on senescent tumor cells may facilitate the binding of innate immune system cells to the tumor cells, leading to their ultimate destruction.

The assay system of the invention can be used to identify additional molecules that are up-regulated in senescent tumor cells and facilitates binding of tumor cells by innate immune system cells.

### REFERENCES AND NOTES

1. B. Vogelstein, D. Lane, A. J. Levine, Nature 408, 307 (2000).
2. S. L. Harris, A. J. Levine, Oncogene 24, 2899 (2005).
3. C. J. Sherr, Cell 116, 235 (2004).
4. S. W. Lowe, E. Cepero, G. Evan, Nature 432, 307 (2004).
5. L. Zender et al., Gastroenterology 123, 608 (2002).
6. F. Staib, S. P. Hussain, L. J. Hofseth, X. W. Wang, C. C. Harris, Hum. Mutat. 21, 201 (2003).
7. R. A. Dickins et al., Nat. Genet. 37, 1289 (2005).
8. L. Zender et al., Cold Spring Harb. Symp. Quant. Biol. 70, 251 (2005).
9. L. Zender et al., Cell 125, 1253 (2006).
10. M. Jain et al., Science 297, 102 (2002).
11. J. S. Fridman, S. W. Lowe, Oncogene 22, 9030 (2003).
12. P. Kahlem, B. Dorken, C. A. Schmitt, J. Clin. Invest 113, 169 (2004).
13. M. Serrano, A. W. Lin, M. E. McCurrach, D. Beach, S. W. Lowe, Cell 88, 593 (1997).
14. M. Braig et al., Nature 436, 660 (2005).
15. Z. Chen et al., Nature 436, 725 (2005).
16. M. Collado et al., Nature 436, 642 (2005).
17. C. Michaloglou et al., Nature 436, 720 (2005).
18. J. Campisi, Cell 120, 513 (2005).
19. T. Minamino et al., Circulation 108, 2264 (2003).
20. D. N. Shelton, E. Chang, P. S. Whittier, D. Choi, W. D. Funk, Curr. Biol. 9, 939 (1999).
21. L. Chin et al., Nature 400, 468 (1999).
22. G. H. Fisher et al., Genes Dev. 15, 3249 (2001).
23. C. M. Shachaf et al., Nature 431, 1112 (2004).
24. V. J. Bykov et al., Nat. Med. 8, 282 (2002).
25. L. T. Vassilev et al., Science 303, 844 (2004).
26. I. B. Roninson, Cancer Res. 63, 2705 (2003).
27. C. A. Schmitt et al., Cell 109, 335 (2002).
28. C. A. Schmitt, Nat. Rev. Cancer 3, 286 (2003).
29. A. Krtolica, S. Parrinello, S. Lockett, P. Y. Desprez, J. Campisi, Proc. Natl. Acad. Sci. U.S.A 98, 12072 (2001).
30. B. Mundt et al., FASEB J 17, 94 (2003).
31. F. Hong et al., Cancer Res. 63, 9023 (2003).
32. M. Narita et al., Cell 113, 703 (2003).
33. D. N., Shelton et al., Microarray analysis of replicative senescence. Curr. Biol. 9, 939-945 (1999).

### Materials and Methods

The following describes in detail the methods and reagents actually used in the experiments described above. These methods and reagents are for illustrative purpose only, and are not limiting in any respect unless specifically provided herein.

Generation of Liver Carcinomas with Reversible p53

Isolation, culture and retroviral infection of murine hepatoblasts were described recently (8, 9). Liver progenitor cells were infected with MSCV retroviruses harboring H-rasV12, a p53 short hairpin RNA driven by the TRE-CMV promoter and tTA. For some experiments the cells were subsequently infected with a luciferase expressing retrovirus and selected with hygromycin. The luciferase-hygro vector was generated by cloning the luciferase cDNA (pGL3, Promega) into the MSCV-Hygro vector (Clontech). Generation of all other vectors has been described recently (7). Genetically modified hepatoblasts were introduced into the livers of retrorsine pretreated (8) female NCR nu/nu mice (6-8 weeks of age) by intra-splenic injection. Transplanted cells were allowed to migrate to the recipient liver and engraft the organ. Tumor progression or regression was monitored by abdominal palpation, whole body GFP imaging and in vivo bioluminescence imaging.

To generate subcutaneous tumors, female nude mice (NCR nu/nu) were γ-irradiated (400 rad) and 3×10⁶ cells (unless otherwise noted in the figure legend) were subcutaneously injected into the rear flanks of the mice. Tumor volume (cm³) was determined by caliper measurement and calculated as 0.52×length×width×width.

### Doxycycline (Dox) Treatment and In Vivo Bioluminescence Imaging

Doxycycline (BD) was refreshed in cell culture medium (100 ng/mL) every 2 days. Mice were treated with 0.2 mg/mL Dox in 0.5% sucrose solution in light-protected bottles. Dox was refreshed every 4 days. Bioluminescence imaging was performed on anaesthetized animals using a Xenogen imager. 200 μL luciferin salt (Xenogen, 15 mg/mL in PBS) was injected into mice (i.p.) 10-15 minutes before imaging. Exposure time was 30 seconds for animals and 10 seconds for explanted livers.

### Tumor Analysis and Immunohistochemistry

Histopathological evaluation of murine liver carcinomas was done by a pathologist using paraffin embedded liver tumor sections stained with Hematoxylin/Eosin. Ki67 and TUNEL staining was performed using standard protocols (2). CK8 (RDI), AFP (Dako), CK7 (Abcam) immunohistochemistry was performed on paraffin embedded liver tumor sections.

### Immunoblotting

Fresh tumor tissue or cell pellets were lysed in Laemmli buffer using a tissue homogenizer. Equal amounts of protein (16 mg) were separated on 10% SDS-polyacrylamide gels and transferred to PVDF membranes. Blots were probed with antibodies against p53 (Vector Laboratories, IMX25, 1:1000), Ras (Calbiochem, Ab1, 11:1000), Tubulin (B-5-1-2, Sigma; 1:5000), AFP (Dako; 1:1000), Cytokeratin 8 (RDI, 1:1000), AFP (Dako, 1:1000), Cytokeratin 7 (Abcam, 1:1000), p15 (Cell signaling, 1:1000), p16 (Santa Cruz, M156, :500) and Dcr2 (Stressgen, 1:2000).

RNA Extraction, Quantitative Real-Time PCR and siRNA Northern Blotting

Murine hepatoma cells or tumors were freshly homogenized in Trizol (GIBCO). RNA was isolated according to the manufacturer's instructions, treated with RNase-free DNase (QIAGEN) and purified with QIAGEN RNAeasy columns. Total RNA was converted to cDNA using TaqMan reverse transcription reagents (Applied Biosystems) and used in qPCR reactions with incorporation of SYBR Green PCR Master Mix (Applied Biosystems). Each reaction was done in triplicate using gene-specific primers. The expression level of each gene was first normalized to AcRP0 (acidic ribosomal protein P0) and then to the first sample (p53 off) among the tumors or the cells. Similar results were obtained using β-actin as reference gene. siRNA northern blotting has been described recently (7).

### Immunofluorescence and Suppression of Immune Cellfunction In Vivo

Sections (10 μm) of snap frozen tumor tissue were fixed with 4% PFA for 10 minutes and subjected to standard immunofluorescence staining using α-Neutrophil (Abcam, NIMP-R14, 1:100) or α-Macrophage (Serotec, CD68 Clone FA-11, 1:100) antibodies together with DAP1 counterstain.

Suppression of macrophage function by GdCl was performed as described recently (30). The neutrophil inhibitory antibody (31) (LY-6G, eBioscience) was injected i.p. (150 mg in 300 μl saline) into mice at d0, d3, d6, d9 with respect to the first day of Dox treatment.

### Colony Formation and SA-β-Gal Assays

Tissue culture, cell counting and colony formation assays were performed as previously described (7). 5,000 cells were plated in 10 cm plates and were stained 8 days or 16 days later. Detection of SA-β-gal activity was performed as described before at pH=5.5 (32). Sections (10 μm) of snap frozen tumor tissue were fixed with 1% formalin for 1 minute and stained for 12-16 hrs. Tumor bearing livers were fixed with 4% formalin overnight, washed with PBS and stained for 4 hrs. Cultured cells were fixed with 4% formalin for 5 minutes and stained for 10 hrs.

Primer Sequences for RT-Q-PCR
Primers for mouse genes used in RT-Q-PCR reactions
were as follows:

| | |
|---|---|
| MCP-1 | |
| 5′-gtggggcgttaactgcat-3′ | (SEQ ID NO: 1) |
| 5′-caggtccctgtcatgcttct-3′ | (SEQ ID NO: 2) |
| CSF-1 | |
| 5′-tgctaggggtggctttagg-3′ | (SEQ ID NO: 3) |
| 5′-caacagctttgctaagtgctcta-3′ | (SEQ ID NO: 4) |
| IL-15 | |
| 5′-cgtgctctaccttgcaaaca-3′ | (SEQ ID NO: 5) |
| 5′-tctcctccagctcctcacat-3′ | (SEQ ID NO: 6) |
| CXCL1 | |
| 5′-tgttgtgcgaaaagaagtgc-3′ | (SEQ ID NO: 7) |
| 5′-tacaaacacagcctcccaca-3′ | (SEQ ID NO: 8) |
| VEGFa | |
| 5′-ggttcccgaaaccctgag-3′ | (SEQ ID NO: 9) |
| 5′-gcagcttgagttaaacgaacg-3′ | (SEQ ID NO: 10) |
| AcRPO | |
| 5′-ttatcagctgcacatcactcag-3′ | (SEQ ID NO: 11) |
| 5′-cgagaagacctccttcttcca-3′ | (SEQ ID NO: 12) |
| β-actin | |
| 5′-ccaccgatccacacagagta-3′ | (SEQ ID NO: 13) |
| 5′-ggctcctagcaccatgaaga-3′ | (SEQ ID NO: 14) |

The practice of the various aspects of the present invention may employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986). All patents, patent applications and references cited herein are incorporated in their entirety by reference.

Those skilled in the art will recognize, or be able to ascertain using no more than routine experimentation, many equivalents to the specific embodiments of the invention described herein. Such equivalents are intended to be encompassed by the following embodiments.

1. A method for making a liver cancer model, said method comprising:
(a) altering hepatocytes:
(1) so as to be capable of modulated tumor suppressor gene expression, said modulation being effected by a controllable inhibition of the expression or function of a tumor suppressor gene in the hepatocytes, and,
(2) to increase oncogene expression, said expression being effected by transducing an oncogene into the hepatocytes;
(b) transplanting said hepatocytes:
(1) into a recipient non-human animal, wherein the hepatocytes engraft the liver of said animal, and a liver cancer develops from at least one of the altered hepatocytes, or,
(2) subcutaneously into a recipient non-human animal, wherein a hepatocellular cancer develops from at least one of the altered hepatocytes.

2. The method of claim 1, wherein the controllable inhibition of the expression or function of the tumor suppressor gene is effected by an antagonist capable of inhibiting the expression or function of the tumor suppressor gene, the antagonist being provided in or added to the hepatocytes.

3. The method of claim 2, wherein the antagonist is an antibody specific for a gene product encoded by the tumor suppressor gene, a polynucleotide encoding a dominant negative mutant of a gene product encoded by the tumor suppressor gene, or a viral oncoprotein that specifically inactivates a gene product encoded by the tumor suppressor gene.

4. The method of claim 2, wherein the antagonist is an siRNA or a precursor molecule thereof.

5. The method of claim 2, wherein the antagonist is synthesized in the hepatocytes under the control of a reversible promoter.

6. The method of claim 5, wherein the reversible promoter is a tetracyclin-responsive promoter.

7. The method of claim 4, wherein the precursor molecule is a precursor microRNA.

8. The method of claim 4, wherein the precursor molecule is a short hairpin RNA (shRNA).

9. The method of claim 4, wherein the siRNA or precursor molecule thereof is encoded by a single copy of nucleic acid construct integrated into the genome of the hepatocytes.

10. The method of claim 1, further comprising, in step (a), altering the hepatocytes to express a fluorescent marker gene.

11. A non-human animal produced by the method of claim 1.

12. A method for determining the effect of increasing the expression of a tumor suppressor gene on the efficacy of a potential therapy or potential therapeutic agent for treating liver cancer, comprising:
(a) administering to a non-human animal, produced by the method of claim 1, the potential therapy or the potential therapeutic agent, under a first condition wherein the expression of the endogenous tumor suppressor gene is decreased from its basal level in the unaltered hepatocytes, and under a second condition wherein the expression of the endogenous tumor suppressor gene is increased from its decreased level; and,
(b) monitoring and comparing the non-human animal for liver tumor formation or growth under the first condition and the second condition,
wherein increased time to tumor formation or growth when the expression of the tumor suppressor gene is increased indicates a positive impact of the tumor suppressor gene on the efficacy of the potential therapy or the potential therapeutic agent.

13. The method of claim 12, wherein the potential therapy is surgery, chemotherapy, radiotherapy, or combination thereof.

14. A method for determining the effect of increasing the expression of a tumor suppressor gene in treating liver cancer, comprising:
(a) allowing tumor formation or growth in a non-human animal produced by the method of claim 1, wherein the expression of an endogenous tumor suppressor gene is decreased from its basal level in the unaltered hepatocytes;
(b) increasing the expression of the endogenous tumor suppressor gene from its decreased level in the altered hepatocytes in the non-human animal; and,
(c) monitoring and comparing the non-human animal for liver tumor growth under conditions (a) and (b),
wherein reduced tumor growth or tumor remission when the expression of the tumor suppressor gene is increased indicates a positive impact of increasing the expression of the tumor suppressor gene in treating liver cancer.

15. A method for determining the role of a gene in liver tumorigenesis, the method comprising:
(a) introducing into a non-human animal an altered hepatocyte comprising a nucleic acid construct encoding an antagonist of the gene, wherein the synthesis of said antagonist is controlled by a reversible promoter; and,
(b) expressing the antagonist such that the altered hepatocyte exhibits decreased expression of the gene as compared to its basal level in the unaltered hepatocyte;
wherein when the altered hepatocyte gives rise to a transfected tumor cell in vivo indicates that the gene negatively regulates liver tumorigenesis.

16. The method of claim 15, wherein the antagonist is an siRNA or precursor molecule thereof.

17. A method for treating a patient having a cancer associated with a deficiency in a tumor suppressor gene, comprising expressing the tumor suppressor gene in the cancer to cause senescence of the majority of the cancer cells.

18. The method of claim 17, further comprising the step of stimulating the innate immune system of the patient.

19. The method of claim 18, wherein the innate immune system of the patient is stimulated by administering to the patient a pharmaceutical composition comprising one or more chemokines.

20. The method of claim 19, wherein the chemokines are CSF1, MCP1, IL-15, or CXCL1.

21. The method of claim 18, wherein macrophages or neutrophils of the innate immune system are activated or stimulated.

22. The method of claim 17, further comprising administering to the patient an angiogenesis inhibitor.

23. The method of claim 17, wherein the tumor suppressor gene is p53.

24. The method of claim 23, wherein p53 is expressed transiently.

25. The method of claim 17, wherein the cancer is liver cancer.

26. An in vitro assay system comprising a co-culture of:
(a) liver tumor cells having:
(1) modulated tumor suppressor gene expression, said modulation being effected by a controllable inhibition of the expression or function of an endogenous tumor suppressor gene in the liver tumor cells, and,
(2) increased oncogene expression effected by a transduced oncogene; and,
(b) innate immune system cells.

27. The in vitro assay system of claim 26, wherein said innate immune system cells comprise macrophages or neutrophils.

28. The in vitro assay system of claim 27, wherein said macrophages or neutrophils are stimulated by one or more cytokines.

29. The in vitro assay system of claim 26, wherein said liver tumor cells are capable of entering senescence upon restoration of the expression or function of the tumor suppressor gene.

30. A screening method to identify a compound that modulates the interaction between innate immune system cells and senescent liver tumor cells, the method comprising:
(a) providing a co-culture of the in vitro assay system of claim 26;
(b) contacting the co-culture with a candidate compound; and,
(c) determining the degree of elimination/killing effect of the senescent liver tumor cells by the innate immune system cells, in the presence and absence of the candidate compound;
wherein an increase (or decrease) of the degree in the presence of the candidate compound indicates that the candidate compound is a positive (or negative) modulator of the interaction between the innate immune system cells and the senescent liver tumor cells.

31. The screening method of claim 30, further comprising inducing, in step (a), the liver tumor cells to undergo senescence by restoring the expression or function of the endogenous tumor suppressor gene.
